SEQUENCE LISTING (22 sequences)

SEQ ID NO: 1 - DNA, 2010 bp, Saccharomyces cerevisiae
  source 1..2010: mol_type = genomic DNA
atgagcaatccccaaaaggcactgaacgacttcctcagctccgagagcgtccacacccacgactcctcacgtaagcagtccaacaagcaatcctcggacgagggccgtagctcgagccagccctcccaccatcactccggaggaactaataacaataacaacaataacaacaacaacaacaacagcaacaacaacaacaacggtaacgatggcggcaacgacgacgactatgactacgagatgcaggactaccgtccctccccacagtctgctaggcctactcccacctacgtgccccaatacagtgtcgagtcgggcaccgcattccccattcaagaggtcattccctcggcctacattaacacccaagacatcaaccacaaggacaacggccccccttccgcatcgtcaaaccgcgctttccgcccccgcggccagacgaccgtcagcgccaacgtcctgaacatagaggacttctacaagaatgcggacgatgcacacacaattccagagtcgcatctgagccggcgtcgctcccgatcacgggccacgtcaaatgcaggccactccgccaacaccggcgccaccaatggtcgtactacgggcgcccagaccaacatggagagcaacgagtcccccaggaacgtgccgataatggtcaagcctaagaccctctaccagaacccgcagacccccacagttctcccctcaacttaccacccgattaacaagtggagctccgttaagaatacctatttaaaagagttcctcgccgagtttatgggtacgatggtcatgatcatcttcggcagcgccgtcgtatgccaggtcaacgtcgccggcaagatccagcaggacaacttcaacgtagccctcgacaacctcaatgtcacgggctcttccgccgagacaatcgacgccatgaagagcctgactagcctagtctcctctgtcgccggtggcaccttcgatgacgttgccctcgggtgggccgctgccgtggtaatgggctacttttgtgcaggtggctcggcaatctctggagcccacctgaacccatcaatcaccctcgctaacctcgtttatcgaggctttcccctgaagaaggtcccatattacttcgcaggccagctgattggtgctttcacgggagccctcattctgttcatttggtacaagagggtccttcaggaggcatactcggactggtggatgaacgaatccgtcgctggcatgttttgtgtcttcccaaagccctacctgagctctgggcggcagttttttagcgagttcctctgcggcgccatgctccaggccgggacattcgccctgacggatccctacacgtgcttgagctcagatgtatttcccctgatgatgttcattctcattttcatcattaacgcttcgatggcatatcagacaggtactgcgatgaacctggcacgggatctgggcccccgcctcgccctctacgcagtgggcttcgaccacaagatgctctgggtccaccaccaccacttcttttgggtgccgatggtagggccgtttattggtgccctgatggggggcctcgtctacgacgtctgtatctaccaaggccacgaatcaccggttaactggtctctccccgtgtacaaggaaatgattatgcgcgcatggtttaggcgccccggatggaagaagcggaaccgagctaggaggacttctgacctctccgacttctcttacaataatgacgacgacgaggaattcggcgaacgcatggcgcttcagaagacgaagacgaagtctagcatttccgacaacgagaacgaggccggcgagaagaaggttcagttcaagtccgttcagcgagggaagcggaccttcggcggcatcccgacaattcttgaggaggaggatagcatcgagactgccagcctcggcgccacgacaacggattctattggtctctctgacactagctctgaggacagccactacggcaacgcgaagaaagtcacctga

SEQ ID NO: 2 - protein, 669 aa, Saccharomyces cerevisiae
MSNPQKALNDFLSSESVHTHDSSRKQSNKQSSDEGRSSSQPSHHHSGGTNNNNNNNNNNNNSNNNNNGNDGGNDDDYDYEMQDYRPSPQSARPTPTYVPQYSVESGTAFPIQEVIPSAYINTQDINHKDNGPPSASSNRAFRPRGQTTVSANVLNIEDFYKNADDAHTIPESHLSRRRSRSRATSNAGHSANTGATNGRTTGAQTNMESNESPRNVPIMVKPKTLYQNPQTPTVLPSTYHPINKWSSVKNTYLKEFLAEFMGTMVMIIFGSAVVCQVNVAGKIQQDNFNVALDNLNVTGSSAETIDAMKSLTSLVSSVAGGTFDDVALGWAAAVVMGYFCAGGSAISGAHLNPSITLANLVYRGFPLKKVPYYFAGQLIGAFTGALILFIWYKRVLQEAYSDWWMNESVAGMFCVFPKPYLSSGRQFFSEFLCGAMLQAGTFALTDPYTCLSSDVFPLMMFILIFIINASMAYQTGTAMNLARDLGPRLALYAVGFDHKMLWVHHHHFFWVPMVGPFIGALMGGLVYDVCIYQGHESPVNWSLPVYKEMIMRAWFRRPGWKKRNRARRTSDLSDFSYNNDDDEEFGERMALQKTKTKSSISDNENEAGEKKVQFKSVQRGKRTFGGIPTILEEEDSIETASLGATTTDSIGLSDTSSEDSHYGNAKKVT

SEQ ID NO: 3 - DNA, 996 bp, Trichoderma reesei
  source 1..996: mol_type = genomic DNA
atgtcttccggaaggaccgtcaccctcaacaccggctacaagatcccccagatcggctacggcacctggcaggccgctcccggcgaggtcggcgctggtgtctttgaggccctcaaggttggctaccgccacctcgacctggccaaggtctacggcaaccagaaggaggttggtgagggcatcaagaaggctcttgctgaggtccccggcctgaagcgcgaggatattttcatcacctccaagctgtggaacaactcccacaagcccgaggacgtcgagcccgctctcgacgacaccctggccgagcttggcctcgactaccttgacctctacctcatccactggcccgttgcctttgctcccggcgccgacctcttccccaagtccgaggacggctccgaggtgcagctcaaccagaatgtgtccattgtccagacctggaaggccatgaccgagctgcccaagtccaaggtccgctccgtcggtgtctccaactttaccatagagcacctcgacgccgtcatcgaggccaccggcgtcgtccccgccgtcaaccagatcgagcgccacccccgcctccccaaccagcctctgatcgactactgcgccaagaagggcatcatcatcaccgcctactccgcctttggcaacaacacaaagggcctgcccctgctcgtcagctccgacgaggtcaaggccgtcgccgacaacctgtccaagaagcagggcaagaccgtcactcccgcccaggtcatcctcgcctggtcccagattggtggccacaccgtcattcccaagtccgtcaccaaggcgcgcattgcggagaacttccaggaggttgagctggatgacgaggccattgctgcgctgaacaagttgggcgagaagcctcagcggttcaacattccttacacctacaagcctaggtggaacattaacctgttcaacaccgaggaggagaaggccgctgcccacactgctgtcatcaagctgtaa

SEQ ID NO: 4 - protein, 331 aa, Trichoderma reesei
MSSGRTVTLNTGYKIPQIGYGTWQAAPGEVGAGVFEALKVGYRHLDLAKVYGNQKEVGEGIKKALAEVPGLKREDIFITSKLWNNSHKPEDVEPALDDTLAELGLDYLDLYLIHWPVAFAPGADLFPKSEDGSEVQLNQNVSIVQTWKAMTELPKSKVRSVGVSNFTIEHLDAVIEATGVVPAVNQIERHPRLPNQPLIDYCAKKGIIITAYSAFGNNTKGLPLLVSSDEVKAVADNLSKKQGKTVTPAQVILAWSQIGGHTVIPKSVTKARIAENFQEVELDDEAIAALNKLGEKPQRFNIPYTYKPRWNINLFNTEEEKAAAHTAVIKL

SEQ ID NO: 5 - DNA, 2055 bp, Trichoderma reesei
  source 1..2055: mol_type = genomic DNA
atgggctacacagaggttgaccagaaggccatcaacaccatccgcctgctcgcggtcgatgccaccttcaagagcaactcgggccacccgggtgcccccatgggcatggctcccgttgcccacgtcctttgggacaagttcatgaggttcaaccccaagaaccccaagtggctgaaccgtgaccgattcgttctctccaacggccacggctgcatgctccagtatgctctgcttcacctgttcggctacgacctgaccatcgatgacctcaaggccttccgaaccgtcgacagcaagactcctggtcaccccgaggcccacgatactcccggtgtcgaggtcaccaccggccccctgggtcagggtatctccaatgccgtcggtctggccatggcccaggcccacaccgccgccaccttcaacaagcccggcttcaccctgtgtgacaactacacctactgcttcctcggcgatggctgcttgatggagggtgtctctgccgaggcctcgtccctggccggccacctccagctcggcaacctcatctgcatctacgatgacaaccacatctccattgacggcgacaccaactgtgccttcaccgaggacgtggccaagcgctacgaggcctacggctggcacgtcgaggtcgtcgagaacggcgaccacgacctcgaggccatggaggccgccatcaagaagtgccaggaggtcaaggacaagccctcaatcatcaagctgcgcaccaccattggcttcggctctctccaggagggcacccacggagtccacggctctcccctcaaggccgacgacatcaagcagctcaagcagaagtttggcttcaaccccgacgagagcttcgtcgtccccaaggaggtctccgacctctacgcccgccactcagccgagggcgccgccgccgaggaggagtggaacaagctgttcgccaagtacgccgagaagtaccccgccgaggctgctgacctccagcgccgcctcaagggcgagctgcccgagggctgggagaagaacctgcccgtctactcgcccagcgatgctgccattgccagccgaaagctgtccgagacggtcctcaccaagatccacgacgtcctgcccgagctccagggtggttccgccgatctgactggttccaacctgacccgctggaaggacgctgttgacttccagccccccgccaccggcctcggtgactactccggacgatacatccgctacggtgtccgcgagcacggcatgggcgccatcatgaacggcctggccgcctacggcaccatcatcccctacggcggtaccttcctcaacttcgtctcctacgccgccggtgccgtccgcctgtccgctctgtcccgcgtccgcgtcatctgggtcgccacccacgactccattggcctgggcgaggacggtcctactcaccagcctatcgagactctggctcacttccgcgccctgcccaactgcatggtgtggcgccccgccgacggcaacgagaccagcgctgcctactacattgccctgacctccaagcacacccccagcatcattgccctgtctcgccagaacctgccccagctcgagggctccgtcatcgagaaggccatcaagggaggatacgtcctgcaggaggtcgagggtgcccagatcaccctggtctccaccggttccgaggtttccatcgccgtggatgccgccaagtacctcctggagaagcacaacgtcaaggcccgcgtcgtctccatgccctgcttcgaggttttcgatgctcagcccaaggagtacagactctccgtcctgcccgacggcatcccctctctgtccattgaggtgatgagcaccatgggctgggagcgatacactcacgagcagtttggtctcaaccgattcggtgcctctggcgcctacaaggatgtctacaagaagttcgagttcacccccgagggcatcaccaagcgcgctcttctcaccattgacttctggaaggacgtccccaacgtccgctctcccatcaaccgtgccttccagcagctcatc

SEQ ID NO: 6 - protein, 685 aa, Trichoderma reesei
MGYTEVDQKAINTIRLLAVDATFKSNSGHPGAPMGMAPVAHVLWDKFMRFNPKNPKWLNRDRFVLSNGHGCMLQYALLHLFGYDLTIDDLKAFRTVDSKTPGHPEAHDTPGVEVTTGPLGQGISNAVGLAMAQAHTAATFNKPGFTLCDNYTYCFLGDGCLMEGVSAEASSLAGHLQLGNLICIYDDNHISIDGDTNCAFTEDVAKRYEAYGWHVEVVENGDHDLEAMEAAIKKCQEVKDKPSIIKLRTTIGFGSLQEGTHGVHGSPLKADDIKQLKQKFGFNPDESFVVPKEVSDLYARHSAEGAAAEEEWNKLFAKYAEKYPAEAADLQRRLKGELPEGWEKNLPVYSPSDAAIASRKLSETVLTKIHDVLPELQGGSADLTGSNLTRWKDAVDFQPPATGLGDYSGRYIRYGVREHGMGAIMNGLAAYGTIIPYGGTFLNFVSYAAGAVRLSALSRVRVIWVATHDSIGLGEDGPTHQPIETLAHFRALPNCMVWRPADGNETSAAYYIALTSKHTPSIIALSRQNLPQLEGSVIEKAIKGGYVLQEVEGAQITLVSTGSEVSIAVDAAKYLLEKHNVKARVVSMPCFEVFDAQPKEYRLSVLPDGIPSLSIEVMSTMGWERYTHEQFGLNRFGASGAYKDVYKKFEFTPEGITKRALLTIDFWKDVPNVRSPINRAFQQLI

SEQ ID NO: 7 - DNA, 975 bp, Trichoderma reesei
  source 1..975: mol_type = genomic DNA
atgagctcctctcttgaccagctcaaggccactggcaccgttgtcgtctctgacactggtgactttgctgccatcgccaagtacaagccccaggatgccaccaccaacccctccctcatcctggccgcctccaagaagcccgagtacgccaagctgatagacgtcgccatcgactacgccaagcagaagggcggcgacattgagcagcaggtcgacgatgccctcgaccgcctgctggtcgagttcggcaaggagattctcaagattgttcccggcaaggtctccaccgaggtcgacgcccggttctcctttgacaccaaggcctctgtcgacaaggccctccacattatcgagctctacaaggagctcggcatccccaaggagcgcgtcctcatcaagatcgcctccacctgggagggcatcaaggccgccgagatcctgcagcgcgaccacggcatcaactgcaacctgacgctcatgttctccctgccccaggccatcggcgccgccgaggccggcgccttcctcatctcccccttcgttggccgcatcctcgactggttcaaggcgagcaccaagaaggactactccaaggaggaggaccccggtgtcgcctccgtcaagagcatcttcaactactacaagaagtttggctacaagaccattgtcatgggtgcctcgttccgcaacacgggcgagattaccgagctcgctggctgcgactacctgaccatttctcccaacctgctcgaggagctcctcaactccaccgagcccgtccccaagaagctcgacgccgccgccgctgcctcccttaacattgagaagaagtcgtacatcaacgacgagtccctgttccgcttcgacttcaacgaggaccagatggccgtcgagaagctgcgcgagggcatcagcaagtttgccgccgacgccgtcaccctcaaggacattctgaggcagaagctgtctgcttaa

SEQ ID NO: 8 - protein, 324 aa, Trichoderma reesei
MSSSLDQLKATGTVVVSDTGDFAAIAKYKPQDATTNPSLILAASKKPEYAKLIDVAIDYAKQKGGDIEQQVDDALDRLLVEFGKEILKIVPGKVSTEVDARFSFDTKASVDKALHIIELYKELGIPKERVLIKIASTWEGIKAAEILQRDHGINCNLTLMFSLPQAIGAAEAGAFLISPFVGRILDWFKASTKKDYSKEEDPGVASVKSIFNYYKKFGYKTIVMGASFRNTGEITELAGCDYLTISPNLLEELLNSTEPVPKKLDAAAAASLNIEKKSYINDESLFRFDFNEDQMAVEKLREGISKFAADAVTLKDILRQKLSA

SEQ ID NO: 9 - DNA, 1615 bp, Trichoderma reesei
  source 1..1615: mol_type = genomic DNA
ctacaaccaccacatcgccaactcttccttctgcaactccaactccttcacttctcttctctgttctactcttctccacctacttcacctaccgcaatcatggccaacgcaactggcaagaaggcagtccactttggcgccggcaacatcggccgtggcttcgttgcttgcttcctccacaactcgggctatgaggtcgtttttgccgacgtcaacggagacctgattgataccatcaactctaccccctcctacaaagtcatcgaggtcggctccgagggcacctcagaaaacgtcatcaccaactaccgggccatcaactctcggactcacgaggaagacctcgttgaggagatccgcactgccgacgtcgtcacctgctccgtcggccccaacatcctcaagttcatcgctcccgtcatcgcaaagggcatcgaccgccgctccgacgacgagacgcccctgcacgtcattgcctgcgagaacgccattggcgccaccgacacgctggccagctacatcaaggatgccaagaacacggatcctgcccgtctcgagacgcaccacctgcgtgcccgctacgccaactcggctattgaccggatcgtgccggcgcaggaccccaatgccggccttgacgtgaagctggagaagttctttgagtgggttgtcgacaagaccccctttgaggacattggcatcccgcccattgagggcatccagtgggtcgacaacctggagcccttcatcgagcgcaagctgtacacggtcaacactggccacgctacggctgcctaccacggctactaccgcagaaagcgcaccgtctacgatgctctccaggacaaggagatcatggctgaggtccgtggcgccctgaccgagaccaagaacctgctcgttgccaagcacggcatcgacgaggaggcccagctcgcctacatgaacaagattatcaagcgcatcggcaacccccatttggaggatgctgtcgagcgtgtcggccgcgctcccctgaggaagctgtcgcgcaaggagcgcttcatcggccccgccgccgaggccgccgagaacgacatgcccgtcaagtacctcctcgacgccatcgagatgacgttccgcttccaggacgtcgagggtgacgacgagtccaaggagctctctaccatcatgtccgagaacacccccgaggacgttgtcgccaaggtctgcggcatcaagccgacggagaagatctaccctcgcctggtcgaggttgtcaagcgtgtgcaggctgacagccaggaggagtaacttgcgatggctcttctggcgttctagctcggcatcactcacggcgctgagtggtgaccggcctactgcatctggctttgggcttcttttgatcttttacttctactttttctcttctacaacttgtgttaagaatgcacgaatgaacggcaaggcgctgggttccgggttaatagagcatcgtctcttttgttttctattttttttgtactctgctagggcattccttgacgggaacaaaaggtgctcgggatatcttcatgggaagcttcagtctatagatggactgagcaggcagcataacgggtcaagataccacatc

SEQ ID NO: 10 - protein, 397 aa, Trichoderma reesei
MANATGKKAVHFGAGNIGRGFVACFLHNSGYEVVFADVNGDLIDTINSTPSYKVIEVGSEGTSENVITNYRAINSRTHEEDLVEEIRTADVVTCSVGPNILKFIAPVIAKGIDRRSDDETPLHVIACENAIGATDTLASYIKDAKNTDPARLETHHLRARYANSAIDRIVPAQDPNAGLDVKLEKFFEWVVDKTPFEDIGIPPIEGIQWVDNLEPFIERKLYTVNTGHATAAYHGYYRRKRTVYDALQDKEIMAEVRGALTETKNLLVAKHGIDEEAQLAYMNKIIKRIGNPHLEDAVERVGRAPLRKLSRKERFIGPAAEAAENDMPVKYLLDAIEMTFRFQDVEGDDESKELSTIMSENTPEDVVAKVCGIKPTEKIYPRLVEVVKRVQADSQEE

SEQ ID NO: 11 - DNA, 1418 bp, Trichoderma reesei
  source 1..1418: mol_type = genomic DNA
cagacaactacaaacaagatggttggcgttgacgcagagattatcgccgatgataccagaatcctgggtcaggatcctttgatccccccggctctgctcatctccgagatccccatgactcccaatgccctccagaccgtcgtcaagggccggagagacgctgcccagatcatcatgggccagaacgacaggctattagtcgtcgtcggtccctgctccatccacgaccccgaagccgcccacgagtacgccgcccgcctcaagcagctctccgagaagctcgccgacgacctctgcatcgtgatgcgcgcctacctggagaagcctcgcacgaccgtcggctggaagggcctgatcaacgatcccgatatcgactcgtccttccagatcaacaagggcctgcgcgtctcccgccggctcttcgtcgacctgacctccaacggcatgcccattgccaccgagatgctcgacaccatctccccccagttcctcgccgactgcatctccgtcggcgccattggcgcgcgaacgaccgagtcccagctgcaccgtgagctcgcttccggcctgtccttccccgtgggcttcaagaacggaaccgacggcagccttggcgtcgccattgatgccattggcgctgccgcggccaagcaccacttcatgggtgtgacgaagcagggcctggctgccattacccgcaccaagggcaacgagcacggcttcgtgatcctgagaggcggaagcaagggccccaactttgacaaggagagcgtccaggcggccaagaagatgctgacggacaagggccagaagctggccattatgattgactgttctcacggcaactctcagaagaaccacaacaaccagcccaaggttgccaaggccattgccgaccagctgcgcgagggcgagcgagccattgtcggcgtcatgatcgagtccaacatcaacgagggcaaccaaaaggtccctgctgagggtccggccggtctcaagcgcggcgttagcatcacggatgcatgcatcaactgggaggacacagtcacggtcctcgaggaccttgccgctgccgtccgcacccgacgcgaggtcaactcgggtgctcctagcgcagaggccaaggtgagcactctggaggaggactagacgtagttttaccggaaggggcgtatgtgtgtacgatgagagagaggaacgaaaagcatgagggtcggtttgttgattttgaacattacgatgcgtgagtttatgcgcgcgaatcctggcttcctccctggccagcgtaaataagaaaagttttagggagagcattcgacacggatatcgagaaattggcgcaccaaaggaacatgtatgaaggcatatattgttggctgtccactttgtcacagacggcgcacacgaaccagtttcag

SEQ ID NO: 12 - protein, 376 aa, Trichoderma reesei
MVGVDAEIIADDTRILGQDPLIPPALLISEIPMTPNALQTVVKGRRDAAQIIMGQNDRLLVVVGPCSIHDPEAAHEYAARLKQLSEKLADDLCIVMRAYLEKPRTTVGWKGLINDPDIDSSFQINKGLRVSRRLFVDLTSNGMPIATEMLDTISPQFLADCISVGAIGARTTESQLHRELASGLSFPVGFKNGTDGSLGVAIDAIGAAAAKHHFMGVTKQGLAAITRTKGNEHGFVILRGGSKGPNFDKESVQAAKKMLTDKGQKLAIMIDCSHGNSQKNHNNQPKVAKAIADQLREGERAIVGVMIESNINEGNQKVPAEGPAGLKRGVSITDACINWEDTVTVLEDLAAAVRTRREVNSGAPSAEAKVSTLEED

SEQ ID NO: 13 - DNA, 3267 bp, Trichoderma reesei
  source 1..3267: mol_type = genomic DNA
atgacaacgctgctcaacaacccgccggattcgggtcccccgccgcgccagattcgcttcgtacataaccagggccagccgccgtccaagcgacgcaggatcaatgcagcgtatgttcttctggggcttctctcgccttgcttcgtctgtcttgccgcgtcacccttggtggcaaatggctgtcctagacagataagtgcaagatggcgcgagcatcaacaccatgcggttaccagtgaagctataagtatgtagagctaacctcgtcttgcgcgcagatgcttgacctgtcgaagacgaaagacgagatgcgccggcgaacgaccgctttgcaccacatgcactaagaacgggcaccaatgcctcggttatcccgaggacaggagagatggcgaggcagcggcaaggacgggcgacgaggaaacgacgccaagggaggatgtcaaccaaggcgaggacgacgtcgcctctagaaaacccgctgcttcggggcttgcgaaccgtccgacgtcctctgggggcagcaatctgcaagctacgcatgctgctgcctctgctgcctctgcttcacggccgccgcggcgcaatatcaagcagactatcgaagtcggcgccgatgccaacccgggcgacgcagagccgcaacagtctcccgtactacatcaccaccaccagagccaccaccagaatcaccatcctcatcatcaagctcatctctcagtcacgtccgatgacttgcccagttcgcccaccctccgccgatcggcccccagccgtcgcatcccctatttccgctactttggctccacggccatcgtccccggcttcaagcaaatggtggtctctgttcgcgaccgtcgccgttcgacggcaggctcacattccggcgcctctcttcattcgacgcccagcggggcgatggccagtggctcggccattgacagcgacattgtccgcgaagacattccctcctacgaccccaacaactccgcccccgttcatcccttaatcattcagctagtcaatacgttttttgttcacgtcgggtgcaattacccctttctcaaacaagacaagtttctgcaacatgtaaaggagaaacgggtcgagccgatacttgtcgatgccgtctgcgccatcgcggcgcgcttctcggaccatcacatcctgacgggcggcaacgacaagatgccccggacagagagaggccaggtctttgcgcaacgggccaggcaggccaccgtcgacactttcccttgcccttctgtgggtgcggtccaggcttgcctgctgatggcatacgagggcttcggggcggcccaggatagcgcgctgtggatgtatctgggcttggccattcgcatggcggttgatctcggtctccaaaagtgcgtgggcgttcaatatcaaggcgaaaaggacccttggtacacccggcatcgaagtcgggccaacgacgatgatcaggacagcccagaggcgcacaaggcggatgccgtcgacgcccctagcccggaggaacagaaggaggtagagcaggagcgcatatataccttctggtccgtcttcatcctggaccggctcatctcctctggcactggacggccggtcacgatccgcgacggcgactacgagctcgcctttcccgaatccagcgtcgacctcgctaccggatggccgaacccatttcctgttctcctccagatcatccacctctacggtcaagtgtgcgatgtgctgaacaatctccacgacgcccaagaccttacccaggacaagtgggatcagctatccgagatggagcatcgactcacgagaatgtacaagcactgggacccgcggcttcagttcaacgtcaacaactttaagacgtacctcggcatgggccagggcaccaactttatcctcctccacttttggttccacgcgctcttcatcatattgcatcaaccgactcttcttacccccttttgcgaactgcggagcgagctccagctgctctcggacagccgtgagctgagcatgagcagcgcaaagaccatctgcgacattttgtcctttgccgacttgatagatccgacgagttttattggcaacccattcacgaatcagcccatttatatcgcggcatgcgcatttctcatggaatcgagcgccaacaatgcatctgagggctcgtccagggagggctcgcctcccactcaagcgagtcgctccaggcaacagacgaagcactttaacaagcagtctcggcattcgcttctcgcgtcggccgccaaccagaattaccagcgatgctacaactccctgcagcagatccaggcgtactggggcggagtaggctatatcctcaccgcgctggaccagaagtcaaagggcaagtgggactgcgagacgtataccgtggaggagtatgagagcaccaagctgccccctcctcggccgtcgattggtgagcagctgtcgcggttcgagaagcagtcgtcgcccaagattgccggctcaccgattgcttggagtctgtccggcacggccaattcgcccaactccagcctgacactcatgtatcaggctctcaacgccagcgcaggcggccacactggattccctccgcctgtgcagtccgtggcgagggctgccaatggttcgccggggagcatcgcgctggaccccattcgacacacgatgccggaagtgggaatgtttacgtcggcggttccgcagccaaacatgtctgccattcgaaactctcctcggcggccatatgctgccaattctatgaatgccgcgggggcgaactcgcggaatcagaatatcatgggcttcgaagacattccggaagagtcgccggatgcaaagggtctgatgtccaattactcaacctctggccagccctacgatggatataatgcatccccgaccagcggcaattcgggcatgggcaattctacagccgtgacgagtgcccccagtggaaacacgacgtcgaatgcgaatacccacaacaataacaacaacaacagcaccaccaccaccagcagcagcaacagcaacagcatgtatttcgcgcaaaacatggcatcatatcaagcctgggggccgatgacgggcaacatggacgccatcacgttcgacagccaggacattgacattgaggcgctggggctgcagcagccggacctgatgagcgggtggctcgagtatcttcccagtgatgtgctggggctgtttgacgagcatggcgagtcggggcagggcggccattga

SEQ ID NO: 14 - protein, 983 aa, Trichoderma reesei
MTTLLNNPPDSGPPPRQIRFVHNQGQPPSKRRRINAACLTCRRRKTRCAGERPLCTTCTKNGHQCLGYPEDRRDGEAAARTGDEETTPREDVNQGEDDVASRKPAASGLANRPTSSGGSNLQATHAAASAASASRPPRRNIKQTIENHHPHHQAHLSVTSDDLPSSPTLRRSAPSRRIPYFRYFGSTAIVPGFKQMVVSVRDRRRSTAGSHSGASLHSTPSGAMASGSAIDSDIVREDIPSYDPNNSAPVHPLIIQLVNTFFVHVGCNYPFLKQDKFLQHVKEKRVEPILVDAVCAIAARFSDHHILTGGNDKMPRTERGQVFAQRARQATVDTFPCPSVGAVQACLLMAYEGFGAAQDSALWMYLGLAIRMAVDLGLQKCVGVQYQGEKDPWYTRHRSRANDDDQDSPEAHKADAVDAPSPEEQKEVEQERIYTFWSVFILDRLISSGTGRPVTIRDGDYELAFPESSVDLATGWPNPFPVLLQIIHLYGQVCDVLNNLHDAQDLTQDKWDQLSEMEHRLTRMYKHWDPRLQFNVNNFKTYLGMGQGTNFILLHFWFHALFIILHQPTLLTPFCELRSELQLLSDSRELSMSSAKTICDILSFADLIDPTSFIGNPFTNQPIYIAACAFLMESSANNASEGSSREGSPPTQASRSRQQTKHFNKQSRHSLLASAANQNYQRCYNSLQQIQAYWGGVGYILTALDQKSKGKWDCETYTVEEYESTKLPPPRPSIGEQLSRFEKQSSPKIAGSPIAWSLSGTANSPNSSLTLMYQALNASAGGHTGFPPPVQSVARAANGSPGSIALDPIRHTMPEVGMFTSAVPQPNMSAIRNSPRRPYAANSMNAAGANSRNQNIMGFEDIPEESPDAKGLMSNYSTSGQPYDGYNASPTSGNSGMGNSTAVTSAPSGNTTNSMYFAQNMASYQAWGPMTGNMDAITFDSQDIDIEALGLQQPDLMSGWLEYLPSDVLGLFDEHGESGQGGH

SEQ ID NO: 15 - DNA, 1773 bp, Trichoderma reesei
  source 1..1773: mol_type = genomic DNA
atgtcaaccagacacatttttacgtcatgtgacggcctcgtcaacaaggccatccgaggcatcattacctacaacccttctctcagcctagacgaaagcaaccgggtcgtctatgacaccacatacgacaagtccaaagtcgtcatcataagcggaggcggctccggacatgagcctgcgtggacaggctatgtcggtcaaaacatgctcgcagcttcggttcaaggagacatctttgccagccccagcacaaagcaaatccttgcagcagtggacgctgttccgagtgacaagggcaccattctggtcatcacaaactacactggtgactgtctgcactttgggctggccaacgagaaagccaatgccaaaggccacaactgccggaccatcatctgcggcgacgatgtctctgtcggcaagaacgggagcatggttggtcgcagaggtttggctggacagatgggtgtcctcaaagtcatgggcggagctgctggtgctggaggctcgctggacgaagtttacaatcttggtgtcgcattctcgcagcagattgtgtctattgcggcaaccttggatcattgccatgttccaggacgtgctgaacatggcatgcttcaagaaggcgttgttgaactcggcaccggccctcataacgaacccggatacaagaaactatcccctgcgccatctgcgtctgatctcgtctcccagattctacaatactgtctgaatgaaaaggatccggaaagaggctacgttcacttcgcccctagtgatgaagtcatactacttctcagcaactacggcggcatatcgcacctggagatgggcgctctggttgacgaaattctcgagcagcttgcaagagactggaagattgagccggtgagggtatgcgccggcttcatcgagacgtcactcaatgctccagccttttctgtctccgtcatcaatgtgactgctgccgcaaagaattgcagttactctgtcgaagagatcaagtccttctttgatgccaggacgacgacgttttgggagtccatgactgggtcgcagacacggcggaggacgagacagcagcagattgttcagcccccggaggaagaggtcaaggcagttgatgagtcaaaagatctgactattgatcccacgctcttagagaagatgttgaggaacgcttgtgaagcgctcgtcaaggctgagccagatctcaccaagtgggataccgtcatgggtgatggagattgcggagagacgctcaggactggagccacatctttgctcacggcccttgattcgggattggcgaaatctgggtcagttgtgagagtactattggaactcgaagacatcgtggagagcaagatgggcggtactctgggcggcatattgggcatcttttttgtctccttgcgtgcagctgtcgagaagaatattggactggcccaaagcccggtcttgctctggggaacagctcttgcaacagctctggagagtctcaggaggtatacgcctgccaaggtcggagatcgtactgtcatggacacactcatcccgtttgctaccgtcatgtgtgagtcgggctttgataacggcgtcaaggcggcacaggatggagcggatgcgaccaagacgatgaagccgagactgggaagagcaacgtatgttggcgtgtcgagtgagaacaaggagttgcctcctgatcccggtgcttggggggccatggtggccattcgcggtttgcgacaaggtctggttgaagcgatctag

SEQ ID NO: 16 - protein, 590 aa, Trichoderma reesei
MSTRHIFTSCDGLVNKAIRGIITYNPSLSLDESNRVVYDTTYDKSKVVIISGGGSGHEPAWTGYVGQNMLAASVQGDIFASPSTKQILAAVDAVPSDKGTILVITNYTGDCLHFGLANEKANAKGHNCRTIICGDDVSVGKNGSMVGRRGLAGQMGVLKVMGGAAGAGGSLDEVYNLGVAFSQQIVSIAATLDHCHVPGRAEHGMLQEGVVELGTGPHNEPGYKKLSPAPSASDLVSQILQYCLNEKDPERGYVHFAPSDEVILLLSNYGGISHLEMGALVDEILEQLARDWKIEPVRVCAGFIETSLNAPAFSVSVINVTAAAKNCSYSVEEIKSFFDARTTTFWESMTGSQTRRRTRQQQIVQPPEEEVKAVDESKDLTIDPTLLEKMLRNACEALVKAEPDLTKWDTVMGDGDCGETLRTGATSLLTALDSGLAKSGSVVRVLLELEDIVESKMGGTLGGILGIFFVSLRAAVEKNIGLAQSPVLLWGTALATALESLRRYTPAKVGDRTVMDTLIPFATVMCESGFDNGVKAAQDGADATKTMKPRLGRATYVGVSSENKELPPDPGAWGAMVAIRGLRQGLVEAI

SEQ ID NO: 17 - DNA, 1326 bp, Trichoderma reesei
  source 1..1326: mol_type = genomic DNA
ttaggtgagggtgtaaccgccatcaacgcgaatatcggcaccggtcacatagcggctggcatcagacagcaggaaagccactgggcccatgagatcctgaggcagacccatcttgccctgaggaatgagggacacccatttcttcttcaggtcaggcttttcatccaggattttctcggtcctagttaaggatttgagttagccggccgaggtcacggctaccgttcaagaacagagaagagtgggttcaactcacaaagccgtcagcatgtatcccggcgagatacagttgactcggatgccagcatgcgcccactcaacggcgagcgaagccgcgagatggcggactgcggccttggaggcgttatagggcctagtccagttcaacccgttagctgtcttatgctctgctcatccatgtctcctttttttctttctttcttttttccgtcgtggaagggactcacgcttgaggctgggggacgttgacaatggcccccgacatgctgcctatcatgaccatgctaccagggctcttcctctccatgaggtgacgagcaaccgatgtagcgaagagatatgtgccgtcgacgttgacgccccagagcttgcgaactctatcaatggggtagttggtggcctcgaagttctcggtgaagccagccgaggtgaccaggttgtcaatcttgccatgttgctggatgatctcggcgatacacgcctcaacggaatcggcatctgagacgtcggcgtaatgcgccgtcactttgggaacgctagaccgggtgtcagccgtctgcctgcaaagccgtgttacgtgcagagcggagcggcagctcactcaccgttttgcgttggggttctccttgagaaaggcatccacgagcagtttcgtctgcttctcggcctcctcctctgtcgctgcgtcagctgtctggcctggggtacgagcgacccggaactggcacgcggaatctcactgttcatgtcgacgagggccaggtcggcgccagagaagaccattccctggccaatcaccagtcccagccctcttgcgccgccggtgacgacaccgacgtttccatcgagggagaagccggcaagcgtaggcttgacatactggccgccggtgccagagacgggctcgctgctcggaagctcatgctccggaggatactcgaactcgacctcggcgtcggttcgagagtggctgccgggcttatcctgccgcggcggcggcacactatggaacttcctcgcagcggccagcggcaggcgtctggcgggcgcggaggcaaaggttcggcgagccgcaagccgcagcat

SEQ ID NO: 18 - protein, 334 aa, Trichoderma reesei
MLRLAARRTFASAPARRLPLAAARKFHSVPPPRQDKPGSHSRTDAEVEFEYPPEHELPSSEPVSGTGGQYVKPTLAGFSLDGNVGVVTGGARGLGLVIGQGMVFSGADLALVDMNSEIPRASSGSLTADTRSSVPKVTAHYADVSDADSVEACIAEIIQQHGKIDNLVTSAGFTENFEATNYPIDRVRKLWGVNVDGTYLFATSVARHLMERKSPGSMVMIGSMSGAIVNVPQPQAPYNASKAAVRHLAASLAVEWAHAGIRVNCISPGYMLTALTEKILDEKPDLKKKWVSLIPQGKMGLPQDLMGPVAFLLSDASRYVTGADIRVDGGYTLT

SEQ ID NO: 19 - DNA, 640 bp, Trichoderma reesei
  source 1..640: mol_type = genomic DNA
tcatgcctcaagatgcccaatctcttccaccttcttcgcactcgcactcgatggatcaaactcataccccagccactcagtagccaacctcctcgcaacttccagcccaacaaccctctcccccatacacaacacctgcgcattattgctctttacactgcgctcaacgctaaagctatcatgtgccgtaaccgctcgtatgcccttgaccttgttggcagaaatggcgacgcctagacctgtgccgcagatgagcagagcgcggtccgctttgccctcggcgacgaggcgggcggcggcggcggcgcgctggggatagggtgtcttgtcggagggctcgtcggatcctacgtcgatgacttggtctacgcgggcgtcgctttcaaagtcttttttgatggtgtttttgtagctgacgccggcgtcctgcggaagcgtgagtgagctcgtgtcagtgactgcccgttctgatgacctagattatgcaatcttgacgatgaagagaccgatagtcatgaagacttatgaagttgacacttgatctcgttgtgatatgagatgtgctggttctggcagccgaaacagaagcacttacatcgcagcccatggcgattctccatttcggtgccatcgtatatgt

SEQ ID NO: 20 - protein, 156 aa, Trichoderma reesei
TYTMAPKWRIAMGCDDAGVSYKNTIKKDFESDARVDQVIDVGSDEPSDKTPYPQRAAAAARLVAEGKADRALLICGTGLGVAISANKVKGIRAVTAHDSFSVERSVKSNNAQVLCMGERVVGLEVARRLATEWLGYEFDPSSASAKKVEEIGHLEA

SEQ ID NO: 21 - DNA, 726 bp, Trichoderma reesei
  source 1..726: mol_type = genomic DNA
tcacgcttcagctacttcctgtatcgtcttgacaaactgctccggatcgtgtccgaaccgccccaggaacaatccgtccagtccgctcttcaacttctcgtagagccccggcccggcactccccccatacaaaaccctcgtcgtccccgccctcctcccaacggactccaaccctctgatcccagcaacaacatcgaggatgtgcttctctccagcaggttgactggcaccaatcgcccatacaggctcatacgccaaaatgacctctgcctcggcgggcacagcattcatgacagcatcaacctgcacccgacactcctcaaccgcgatatgcacgtcgccttgagtcttttctccaatgcacacaagcggtatcatgccattgcgacttacagcggcagccttattcgcaaccatggtgtcgtcctcgccaaagatgctcctgcgctctgcatgtccgacttccacgagcttgacgcctgcgctgcgcaacacggaggggctgacttcgccggtgaatgcgccttggtcttcccagtggcagtcttgagcgccggtccaaatggcaacggagctggacttaagctgggcgattgtggtggtcagggagacgaagtcggggatgatgaagatgtcaatgtgggagaggagctctatcgggacgtctgccagcttggataagaagctgtttgtgaagtctcttgtttgctggagagaaaagtacat

SEQ ID NO: 22 - protein, 241 aa, Trichoderma reesei
MYFSLQQTRDFTNSFLSKLADVPIELLSHIDIFIIPDFVSLTTTIAQLKSSSVAIWTGAQDCHWEDQGAFTGEVSPSVLRSAGVKLVEVGHAERRSIFGEDDTMVANKAAAVSRNGMIPLVCIGEKTQGDVHIAVEECRVQVDAVMNAVPAEAEVILAYEPVWAIGASQPAGEKHILDVVAGIRGLESVGRRAGTTRVLYGGSAGPGLYEKLKSGLDGLFLGRFGHDPEQFVKTIQEVAEA

(19) United States
(12) Patent Application Publication
(10) Pub. No.: US 2025/0346930 A1
(43) Pub. Date: Nov. 13, 2025

(54) METHOD FOR THE BIOTECHNOLOGICAL PRODUCTION OF ERYTHRITOL

(71) Applicant: CONZIL ESTATE GMBH, Bodman-Ludwigshafen (DE)

(72) Inventors: Astrid MACH-AIGNER, Wien (AT); Robert MACH, Wien (AT); Audrey MASI, Wien (AT); Laura Vanessa DAZA SERNA, Wien (AT); Anton FRIEDL, Wien (AT); Georg STARK, Wien (AT)

(21) Appl. No.: 18/723,021

(22) PCT Filed: Dec. 15, 2022
(86) PCT No.: PCT/EP2022/086137

§ 371 Date: Jan. 3, 2025

(30) Foreign Application Priority Data
Dec. 21, 2021 (EP) 21216448.7

(51) Int. Cl. C12P 7/18; C12N 1/14; C12N 9/04; C12R 1/885

(52) U.S. Cl. CPC C12P 7/18; C12N 1/145; C12N 9/0006; C12P 2201/00; C12R 2001/885; C12Y 202/01001

(57) ABSTRACT

The present invention pertains to a method for the biotechnological production of erythritol, in particular a method for the biotechnological production of erythritol by cultivating at least one saprotroph in a culture medium comprising a nitrogen source and a high concentration of a carbon source.

The present invention pertains to a method for the biotechnological production of erythritol, in particular a method for the biotechnological production of erythritol by cultivating at least one saprotroph in a culture medium comprising a nitrogen source and a high concentration of a carbon source.

In the recent years people's lifestyle and the growing consumption of food products with high sugar content has resulted in a tremendous rise of blood glucose related diseases and disorders, such as diabetes mellitus type 2 (DMT2). Nowadays, a low-glycaemic nutrition and the avoidance of excessive peaks in blood glucose level is considered to reduce the risk for developing certain chronic diseases and to be beneficial for maintenance and improvement of health and for the treatment and/or prevention of a large number of blood glucose related diseases and disorders.

Erythritol is a naturally occurring four-carbon sugar alcohol gaining increasing importance in the food industry due to its specific properties and its manifold fields of application. It can be found in several fruits, such as pears, grapes and melons, mushrooms, alcoholic drinks (beer, wine, sake) and fermented food products, such as soy sauce and miso bean paste, but naturally also occurs in biofluids of humans and animals such as eye lens tissue, serum, plasma, fetal fluid and urine. Due to its small molecular weight, erythritol is easily absorbed already in the upper intestine and therefore causes less digestive distress than other sugar alcohols used in the food industry. The majority of ingested erythritol is not metabolized in the human body and is excreted unmodified into the urine without changing blood glucose and insulin levels (Regnat et al., Erythritol as sweetener—wherefrom and whereto?, 2018, Applied Microbiology and Biotechnology, Vol. 102). Furthermore, erythritol is non-cariogenic, thermally stable, crystalizes well and is less hygroscopic than sucrose. Due to the negative enthalpy of dissolution, the consumption of erythritol causes a cooling sensation in the oral cavity. A 10% (w/v) solution of erythritol has 60-80% of the sweetness of sucrose at the same concentration.

However, in contrast to other sugar alcohols, such as sorbitol, xylitol, mannitol, lactitol, and maltitol, which are well-established as sugar alternatives for many years, so far erythritol cannot be chemically produced in a commercially worthwhile way. The production of erythritol from dialdehyde starch using a nickel catalyst at high temperatures results in unsatisfying low yields (Moon et al. Biotechnological production of erythritol and its applications, 2010, Appl. Microbiol. Biotechnol., Vol. 86).

In yeast and fungus species, erythritol is produced via the so-called pentose phosphate pathway. It is synthesized from D-erythrose-4-phosphate through dephosphorylation and subsequent reduction of erythrose. Based thereon, the suitability of osmophilic yeast, such as Aureobasidium sp., Trichosporonoides sp. and Candida magnoliae, for the biotechnological production of erythritol has been investigated in several studies (Ishizuka et al., Breeding of a mutant of Aureobasidium sp. with high erythritol production, 1989, J. Ferm. Bioeng., Vol. 68 (5); U.S. Pat. Nos. 4,939,091A; 5,962,287 A; Oh et al., Increased erythritol production in fedbatch cultures of Torula sp. by controlling glucose concentration, 2001, JIM&B, Vol. 26; Koh et al., Scale-up of erythritol production by an osmophilic mutant of Candida magnoliae, 2003, Biotechnol. Lett., Vol. 25; Ryu et al., Optimization of erythritol production by Candida magnoliae in fed-batch culture, 2000, JIM&B, Vol. 25). More recent studies examined the potential of filamentous fungi to produce erythritol (Jovanovic et al., 2013). However, the yields of erythritol obtained from the different strains was unsatisfactory for industrial scale production.

Accordingly, there is a need for a biotechnological method for the production of erythritol with increased yield. The present invention overcomes the disadvantages of the methods in the prior art by the subject-matter of the independent claims, in particular by the method for the production of erythritol according to the present invention.

The present invention in particular pertains to a method for the production of erythritol, comprising the steps:
a) cultivating at least one saprotroph in a culture medium comprising a carbon source in a concentration of at least 40 g/L and a nitrogen source, so as to obtain erythritol in the culture medium,
b) recovering erythritol from the culture medium.

In a preferred embodiment of the present invention, the culture medium comprises the carbon source in a concentration of at least 50 g/L, preferably at least 60 g/L, preferably at least 70 g/L.

In a further preferred embodiment of the present invention, the culture medium comprises the carbon source in a concentration of at most 150 g/L, preferably at most 125 g/L, preferably at most 100 g/L, preferably at most 95 g/L, preferably at most 90 g/L.

According to a preferred embodiment of the present invention, the culture medium comprises the carbon source in a concentration of 40 to 200 g/L, preferably 45 to 175 g/L, preferably 50 to 150 g/L, preferably 55 to 125 g/L, preferably 60 to 100 g/L, preferably 65 to 95 g/L, preferably 70 to 90 g/L.

Preferably, the culture medium comprises the carbon source in a concentration of 40 to 160 g/L, preferably 40 to 150 g/L, preferably 40 to 140 g/L, preferably 45 to 130 g/L, preferably 45 to 120 g/L, preferably 45 to 110 g/L, preferably 50 to 100 g/L, preferably 50 to 95 g/L, preferably 50 to 90 g/L, preferably 60 to 90 g/L, preferably 65 to 90 g/L, preferably 70 to 90 g/L.

In a further preferred embodiment of the present invention, the culture medium comprises the nitrogen source in a concentration of at least 50 mM, preferably at least 55 mM, preferably at least 60 mM, preferably at least 65 mM, preferably at least 70 mM.

Preferably, the culture medium comprises the nitrogen source in a concentration of at most 150 mM, preferably at most 140 mM, preferably at most 130 mM, preferably at most 120 mM, preferably at most 110 mM, preferably at most 100 mM.

Particularly preferred, the culture medium comprises the nitrogen source in a concentration of 50 to 140 mM, preferably 55 to 130 mM, preferably 60 to 120 mM, preferably 65 to 110 mM, preferably 70 to 100 mM.

According to preferred embodiment of the present invention, the culture medium is a synthetic medium. Preferably, the culture medium is not a synthetic medium.

In a further preferred embodiment of the present invention, the culture medium comprises a hydrolysate obtained by hydrothermal treatment of a cellulose-, hemi-cellulose- and/or starch-comprising raw material.

Particularly preferred, the culture medium comprises a lignocellulose-comprising hydrolysate.

In a further preferred embodiment of the present invention, the hydrolysate, preferably the hydrolysate obtained by hydrothermal treatment of a cellulose-, hemi-cellulose- and/or starch-comprising raw material, in particular the lignocellulose-comprising hydrolysate, is derived from agro-industrial residues.

According to preferred embodiment of the present invention, the culture medium comprises a hydrolysate of straw, in particular a hydrolysate of wheat straw. In a further preferred embodiment, the culture medium comprises a hydrolysate of wheat bran. Preferably, the culture medium comprises a hydrolysate of potato pulp.

In a preferred embodiment of the present invention, the carbon source is a monomeric or oligomeric C-5 and/or C-6 sugar or a mixture thereof. Preferably, the carbon source is a monomeric or oligomeric C-5 and/or C-6 sugar selected from arabinose, xylose, glucose, galactose, mannose and fructose. According to a further preferred embodiment, the carbon source is a mixture of monomeric and oligomeric C-5 and/or C-6 sugars, preferably a mixture of monomeric and oligomeric C-5 and/or C-6 sugars selected from arabinose, xylose, glucose, galactose, mannose and fructose.

In a further preferred embodiment, the carbon source is glucose or xylose. Particularly preferred, the carbon source is glucose. Preferably, the carbon source is xylose.

According to a preferred embodiment, the C-5 sugar content in the culture medium amounts to at least 5%, preferably at least 10%, preferably at least 15%, preferably at least 20%, preferably at least 25%, preferably at least 30%, preferably at least 40%, preferably at least 50%, preferably at least 60%, preferably at least 70%, preferably at least 80%, preferably at least 85%, preferably at least 90%, preferably at least 95% (based on total carbohydrates in the culture medium).

Preferably, the C-6 sugar content in the culture medium amounts to at least 10%, preferably at least 15%, preferably at least 20%, preferably at least 25%, preferably at least 30%, preferably at least 40%, preferably at least 50%, preferably at least 60%, preferably at least 70%, preferably at least 80%, preferably at least 85%, preferably at least 90%, preferably at least 95% (based on total carbohydrates in the culture medium).

In a preferred embodiment of the present invention, the glucose content in the culture medium amounts to at least 10%, preferably at least 15%, preferably at least 20%, preferably at least 25%, preferably at least 30%, preferably at least 40%, preferably at least 50%, preferably at least 60%, preferably at least 70%, preferably at least 80%, preferably at least 85%, preferably at least 90%, preferably at least 95% (based on total carbohydrates in the culture medium).

In another preferred embodiment, the xylose content in the culture medium amounts to at least 10%, preferably at least 15%, preferably at least 20%, preferably at least 25%, preferably at least 30%, preferably at least 40%, preferably at least 50%, preferably at least 60%, preferably at least 70%, preferably at least 80%, preferably at least 85%, preferably at least 90%, preferably at least 95% (based on total carbohydrates in the culture medium).

Preferably, the content of monomeric C-5 and C-6 sugars in the culture medium amounts to at least 1%, preferably at least 2%, preferably at least 3%, preferably 4%, preferably at least 5%, preferably at least 6%, preferably at least 7%, preferably at least 8%, preferably at least 9%, preferably at least 10%, preferably at least 15%, preferably at least 20%, preferably at least 30%, preferably at least 40%, preferably at least 50%, preferably at least 60%, preferably at least 70%, preferably at least 80%, preferably at least 85%, preferably at least 90%, preferably at least 95%, preferably at least 96%, preferably at least 97%, preferably at least 98%, preferably at least 99% (based on total carbohydrates in the culture medium).

According to a preferred embodiment of the present invention, the content of oligomeric C-5 and C-6 sugars in the culture medium amounts to at least 10%, preferably at least 20%, preferably at least 30%, preferably at least 40%, preferably at least 50%, preferably at least 60%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99% (based on total carbohydrates in the culture medium).

According to a preferred embodiment of the present invention, the nitrogen source is selected from ammonium salts, nitrate salts, yeast extract and urea. Particularly preferred, the nitrogen source is an ammonium salt or urea. Preferably, the ammonium salt is selected from ammonium sulfate, ammonium nitrate, ammonium citrate, ammonium succinate, ammonium carbonate, ammonium oxalate, and ammonium malate.

In a particularly preferred embodiment of the present invention, the nitrogen source is urea.

Preferably, the culture medium comprises urea in a concentration of at least 8 mM, preferably at least 10 mM, preferably at least 15 mM, preferably at least 20 mM, preferably at least 25 mM, preferably at least 30 mM, preferably at least 35 mM, preferably at least 40 mM, preferably at least 45 mM, preferably at least 50 mM, preferably at least 55 mM, preferably at least 60 mM, preferably at least 65 mM, preferably at least 70 mM, preferably at least 80 mM.

According to a preferred embodiment of the present invention, the culture medium comprises urea in a concentration of at most 160 mM, preferably at most 150 mM, preferably at most 140 mM, preferably at most 130 mM, preferably at most 120 mM, preferably at most 110 mM, preferably at most 100 mM, preferably at most 90 mM.

In a preferred embodiment, the culture medium comprises urea in a concentration of 8 to 160 mM, preferably 10 to 150 mM, preferably 20 to 140 mM, preferably 30 to 130 mM, preferably 40 to 120 mM, preferably 50 to 110 mM, preferably 60 to 100 mM, preferably 65 to 100 mM, preferably 70 to 100 mM, most preferably 70 to 90 mM.

According to a further preferred embodiment of the present invention the at least one saprotroph is a filamentous fungus. Preferably, the filamentous fungus is selected from the group consisting of the genera Hypocrea, Trichoderma, Gibberella, Fusarium, Aspergillus and Penicillium.

In a preferred embodiment, the saprotroph is selected from the group consisting of Hypocrea jecorina (Trichoderma reesei), Gibberella zeae, Fusarium graminearum, Aspergillus niger, Aspergillus nidulans, Aspergillus oryzae and Penicillium chrysogenum. Particularly preferred, the saprotroph is Hypocrea jecorina (Trichoderma reesei).

According to a preferred embodiment of the present invention, the at least one saprotroph is a naturally occurring saprotroph, in particular is not a genetically modified saprotroph.

In another preferred embodiment of the present invention, the at least one saprotroph is genetically modified.

In a particularly preferred embodiment of the present invention, the genetically modified saprotroph is Hypocrea jecorina (Trichoderma reesei). Preferably, the genetically modified saprotroph is based on the Trichoderma reesei strain QM6aΔtmus53.

Preferably, the at least one genetically modified saprotroph comprises at least one gene encoding at least one membrane-bound alditol transporter, at least one gene encoding at least one erythrose reductase and at least one inactivated gene encoding mannitol 1-phosphate 5-dehydrogenase.

In a further preferred embodiment of the present invention, the at least one gene of the genetically modified saprotroph encoding at least one membrane-bound alditol transporter is fps, in particular codon-optimized fps1. Preferably, the at least one gene of the genetically modified saprotroph encoding at least one membrane-bound alditol transporter is fps/from Saccharomyces cerevisiae, in particular is codon-optimized fps1 from Saccharomyces cerevisiae.

In a further preferred embodiment, the at least one gene of the genetically modified saprotroph encoding at least one membrane-bound alditol transporter comprises the nucleotide sequence of SEQ ID No. 1, in particular consists of the nucleotide sequence of SEQ ID No. 1.

In a preferred embodiment of the present invention, the at least one gene of the genetically modified saprotroph encoding at least one membrane-bound alditol transporter comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 1. Preferably, the at least one gene of the genetically modified saprotroph encoding at least one membrane-bound alditol transporter consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 1.

Preferably, the membrane-bound alditol transporter of the genetically modified saprotroph comprises the amino acid sequence of SEQ ID No. 2, in particular consists of the amino acid sequence of SEQ ID No. 2.

Preferably, the membrane-bound alditol transporter of the genetically modified saprotroph comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 2. In a further preferred embodiment, the membrane-bound alditol transporter of the genetically modified saprotroph consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 2.

In another preferred embodiment of the present invention, the membrane-bound alditol transporter of the genetically modified saprotroph comprises an amino acid sequence as defined in SEQ ID No. 2, in which one, preferably two, preferably three, preferably five, preferably six, preferably seven, preferably eight, preferably nine, preferably ten, amino acids are exchanged by other naturally occurring amino acids. In a particularly preferred embodiment, the membrane-bound alditol transporter of the genetically modified saprotroph comprises an amino acid sequence as defined in SEQ ID No. 2, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one gene of the genetically modified saprotroph encoding at least one erythrose reductase is err1, in particular is codon-optimized err1. Preferably, the at least one gene of the genetically modified saprotroph encoding at least one erythrose reductase is err1, in particular is codon-optimized err1, from Trichoderma reesei, Aspergillus niger or Fusarium graminearum.

In a preferred embodiment, the at least one gene of the genetically modified saprotroph encoding at least one erythrose reductase comprises the nucleotide sequence of SEQ ID No. 3, in particular consist of the nucleotide sequence of SEQ ID No. 3.

Preferably, the at least one gene of the genetically modified saprotroph encoding at least one erythrose reductase comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 3. In another preferred embodiment of the present invention, the at least one gene of the genetically modified saprotroph encoding at least one gene encoding at least one erythrose reductase consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 3.

Preferably, the erythrose reductase of the genetically modified saprotroph comprises the amino acid sequence of SEQ ID No. 4, in particular consists of the amino acid sequence of SEQ ID No. 4.

In a preferred embodiment, the erythrose reductase of the genetically modified saprotroph comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 4. Particularly preferred, the erythrose reductase of the genetically modified saprotroph consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 4.

Preferably, the erythrose reductase of the genetically modified saprotroph comprises an amino acid sequence as defined in SEQ ID No. 4, in which one, preferably two, preferably three, preferably five, preferably six, preferably seven, preferably eight, preferably nine, preferably ten, amino acids are exchanged by other naturally occurring amino acids. In a preferred embodiment of the present invention, the erythrose reductase of the genetically modified saprotroph comprises an amino acid sequence as defined in SEQ ID No. 4, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one inactivated gene encoding mannitol 1-phosphate 5-dehydrogenase of the genetically modified saprotroph is mpdh.

Preferably, the at least one gene encoding mannitol 1-phosphate 5-dehydrogenase of the genetically modified saprotroph comprised the nucleotide sequence of SEQ ID No. 9 before inactivation, in particular consisted of the nucleotide sequence of SEQ ID No. 9 before inactivation.

Preferably, the at least one gene encoding mannitol 1-phosphate 5-dehydrogenase of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 9 before inactivation. In another preferred embodiment, the at least one gene encoding mannitol 1-phosphate 5-dehydrogenase of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 9 before inactivation.

Preferably, the mannitol 1-phosphate 5-dehydrogenase encoded by the nucleotide sequence of SEQ ID No. 9 before inactivation comprises the amino acid sequence of SEQ ID No. 10, in particular consists of the amino acid sequence of SEQ ID No. 10.

In a preferred embodiment, the mannitol 1-phosphate 5-dehydrogenase encoded by the nucleotide sequence of SEQ ID No. 9 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 10. Particularly preferred, the mannitol 1-phosphate 5-dehydrogenase encoded by the nucleotide sequence of SEQ ID No. 9 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 10.

Preferably, the mannitol 1-phosphate 5-dehydrogenase encoded by the nucleotide sequence of SEQ ID No. 9 before inactivation comprises an amino acid sequence as defined in SEQ ID No. 10, in which one, preferably two, preferably three, preferably five, preferably six, preferably seven, preferably eight, preferably nine, preferably ten, amino acids are exchanged by other naturally occurring amino acids. In a preferred embodiment of the present invention, the mannitol 1-phosphate 5-dehydrogenase encoded by the nucleotide sequence of SEQ ID No. 9 before inactivation comprises an amino acid sequence as defined in SEQ ID No. 10, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a further preferred embodiment of the present invention, the at least one inactivated gene encoding mannitol 1-phosphate 5-dehydrogenase of the genetically modified saprotroph is deleted.

In a preferred embodiment of the present invention, the at least one inactivated gene encoding mannitol 1-phosphate 5-dehydrogenase of the genetically modified saprotroph is non-functional.

In a particularly preferred embodiment, the at least one inactivated gene encoding mannitol 1-phosphate 5-dehydrogenase of the genetically modified saprotroph is inactivated by gene knock-out, in particular gene replacement. Preferably, the at least one inactivated gene encoding mannitol 1-phosphate 5-dehydrogenase of the genetically modified saprotroph is inactivated by gene replacement using a deletion cassette.

In a preferred embodiment of the present invention, the at least one gene encoding mannitol 1-phosphate 5-dehydrogenase of the genetically modified saprotroph is inactivated, in particular made non-functional, by genome editing, in particular by meganucleases, zinc-finger nucleases (ZFNs), transcription activator-like effector nucleases (TALENs) or by the clustered regularly interspaced short palindromic repeats (CRISPR) system.

In a particularly preferred embodiment, the genetically modified saprotroph comprises at least one, preferably at least two, preferably at least three, preferably at least four, preferably at least five, further inactivated genes. Preferably, the at least one further inactivated gene of the genetically modified saprotroph is selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase 1 (EYI1) and a gene encoding erythritol isomerase 2 (EYI2).

In a preferred embodiment of the present invention, the at least one further inactivated gene of the genetically modified saprotroph is a gene encoding phospho-2-dehydro-3-deoxyheptonate aldolase 1 (Dhaps-1). Preferably, the at least one gene encoding phospho-2-dehydro-3-deoxyheptonate aldolase 1 (Dhaps1) of the genetically modified saprotroph comprised the nucleotide sequence of SEQ ID No. 11 before inactivation, in particular consisted of the nucleotide sequence of SEQ ID No. 11 before inactivation.

Preferably, the at least one gene encoding phospho-2-dehydro-3-deoxyheptonate aldolase 1 (Dhaps1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 11 before inactivation. In further preferred embodiment, the at least one gene encoding phospho-2-dehydro-3-deoxyheptonate aldolase 1 (Dhaps1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 11 before inactivation.

Preferably, the phospho-2-dehydro-3-deoxyheptonate aldolase 1 (Dhaps1) encoded by the nucleotide sequence of SEQ ID No. 11 before inactivation comprises the amino acid sequence of SEQ ID No. 12, in particular consists of the amino acid sequence of SEQ ID No. 12.

In a preferred embodiment, the phospho-2-dehydro-3-deoxyheptonate aldolase 1 (Dhaps1) encoded by the nucleotide sequence of SEQ ID No. 11 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 12. Particularly preferred, the phospho-2-dehydro-3-deoxyheptonate aldolase 1 (Dhaps1) encoded by the nucleotide sequence of SEQ ID No. 11 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 12.

Preferably, the phospho-2-dehydro-3-deoxyheptonate aldolase 1 (Dhaps1) encoded by the nucleotide sequence of SEQ ID No. 11 before inactivation comprises an amino acid sequence as defined in SEQ ID No. 12, in which one, preferably two, preferably three, preferably five, preferably six, preferably seven, preferably eight, preferably nine, preferably ten, amino acids are exchanged by other naturally occurring amino acids. In a preferred embodiment of the present invention, the phospho-2-dehydro-3-deoxyheptonate aldolase 1 (Dhaps1) encoded by the nucleotide sequence of SEQ ID No. 11 before inactivation comprises an amino acid sequence as defined in SEQ ID No. 12, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In another embodiment of the present invention, the at least one further inactivated gene of the genetically modified saprotroph is a gene encoding erythritol utilization factor (EUF). Preferably, the at least one gene encoding erythritol utilization factor (Euf1) of the genetically modified saprotroph comprised the nucleotide sequence of SEQ ID No. 13 before inactivation, in particular consisted of the nucleotide sequence of SEQ ID No. 13 before inactivation.

Preferably, the at least one gene encoding erythritol utilization factor (Euf1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 13 before inactivation. Particularly preferred, the at least one gene encoding erythritol utilization factor (Euf1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 13 before inactivation.

Preferably, the erythritol utilization factor (Euf1) encoded by the nucleotide sequence of SEQ ID No. 13 before inactivation comprises the amino acid sequence of SEQ ID No. 14, in particular consists of the amino acid sequence of SEQ ID No. 14.

In a preferred embodiment, the erythritol utilization factor (Euf1) encoded by the nucleotide sequence of SEQ ID No. 13 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 14. Particularly preferred, the erythritol utilization factor (Euf1) encoded by the nucleotide sequence of SEQ ID No. 13 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 14.

Preferably, the erythritol utilization factor (Euf1) encoded by the nucleotide sequence of SEQ ID No. 13 before inactivation comprises an amino acid sequence as defined in SEQ ID No. 14, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol utilization factor (Euf1) encoded by the nucleotide sequence of SEQ ID No. 13 before inactivation comprises an amino acid sequence as defined in SEQ ID No. 14, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a particularly preferred embodiment of the present invention, the at least one further inactivated gene of the genetically modified saprotroph is a gene encoding erythrulose kinase (EYK1). Preferably, the at least one gene encoding erythrulose kinase (Eyk1) of the genetically modified saprotroph comprised the nucleotide sequence of SEQ ID No. 15 before inactivation, in particular consisted of the nucleotide sequence of SEQ ID No. 15 before inactivation.

According to another preferred embodiment of the present invention, the at least one gene encoding erythrulose kinase (Eyk1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 15 before inactivation. Particularly preferred, the at least one gene encoding erythrulose kinase (Eyk1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 15 before inactivation.

Preferably, the erythrulose kinase (Eyk1) encoded by the nucleotide sequence of SEQ ID No. 15 before inactivation comprises the amino acid sequence of SEQ ID No. 16, in particular consists of the amino acid sequence of SEQ ID No. 16.

In a preferred embodiment, the erythrulose kinase (Eyk1) encoded by the nucleotide sequence of SEQ ID No. 15 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 16. Particularly preferred, the erythrulose kinase (Eyk1) encoded by the nucleotide sequence of SEQ ID No. 15 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 16.

Preferably, the erythrulose kinase (Eyk1) encoded by the nucleotide sequence of SEQ ID No. 15 before inactivation comprises an amino acid sequence as defined in SEQ ID No. 16, in which one, preferably two, preferably three, preferably five, preferably six, preferably seven, preferably eight, preferably nine, preferably ten, amino acids are exchanged by other naturally occurring amino acids. In a preferred embodiment of the present invention, the erythrulose kinase (Eyk1) encoded by the nucleotide sequence of SEQ ID No. 15 before inactivation comprises an amino acid sequence as defined in SEQ ID No. 16, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one further inactivated gene of the genetically modified saprotroph is a gene encoding erythritol dehydrogenase (EYD1). Preferably, the at least one gene encoding erythritol dehydrogenase (Eyd1) of the genetically modified saprotroph comprised the nucleotide sequence of SEQ ID No. 17 before inactivation, in particular consisted of the nucleotide sequence of SEQ ID No. 17 before inactivation.

Preferably, the at least one gene encoding erythritol dehydrogenase (Eyd1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 17 before inactivation. In another preferred embodiment of the present invention, the at least one gene encoding erythritol dehydrogenase (Eyd1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 17 before inactivation.

Preferably, the erythritol dehydrogenase (Eyd1) encoded by the nucleotide sequence of SEQ ID No. 17 before inactivation comprises the amino acid sequence of SEQ ID No. 18, in particular consists of the amino acid sequence of SEQ ID No. 18.

In a preferred embodiment, the erythritol dehydrogenase (Eyd1) encoded by the nucleotide sequence of SEQ ID No. 17 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 18. Particularly preferred, the erythritol dehydrogenase (Eyd1) encoded by the nucleotide sequence of SEQ ID No. 17 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 18.

Preferably, the erythritol dehydrogenase (Eyd1) encoded by the nucleotide sequence of SEQ ID No. 17 before inactivation comprises an amino acid sequence as defined in SEQ ID No. 18, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol dehydrogenase (Eyd1) encoded by the nucleotide sequence of SEQ ID No. 17 before inactivation comprises an amino acid sequence as defined in SEQ ID No. 18, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one further inactivated gene of the genetically modified saprotroph is a gene encoding erythritol isomerase 1 (EYI1). Preferably, the at least one gene encoding erythritol isomerase 1 (Eyi1) of the genetically modified saprotroph comprised the nucleotide sequence of SEQ ID No. 19 before inactivation, in particular consisted of the nucleotide sequence of SEQ ID No. 19 before inactivation.

Particularly preferred, the at least one gene encoding erythritol isomerase 1 (Eyi1) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 19 before inactivation. In a further preferred embodiment of the present invention, the at least one gene encoding erythritol isomerase 1 (Eyi1) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 19 before inactivation.

Preferably, the erythritol isomerase 1 (Eyi1) encoded by the nucleotide sequence of SEQ ID No. 19 before inactivation comprises the amino acid sequence of SEQ ID No. 20, in particular consists of the amino acid sequence of SEQ ID No. 20.

In a preferred embodiment, the erythritol isomerase 1 (Eyi1) encoded by the nucleotide sequence of SEQ ID No. 19 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 20. Particularly preferred, the erythritol isomerase 1 (Eyi1) encoded by the nucleotide sequence of SEQ ID No. 19 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 20.

Preferably, the erythritol isomerase 1 (Eyi1) encoded by the nucleotide sequence of SEQ ID No. 19 before inactivation comprises an amino acid sequence as defined in SEQ ID No. 20, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol isomerase 1 (Eyi1) encoded by the nucleotide sequence of SEQ ID No. 19 before inactivation comprises an amino acid sequence as defined in SEQ ID No. 20, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In another preferred embodiment of the present invention, the at least one further inactivated gene of the genetically modified saprotroph is a gene encoding erythritol isomerase 2 (EYI2). Preferably, the at least one gene encoding erythritol isomerase 2 (Eyi2) of the genetically modified saprotroph comprised the nucleotide sequence of SEQ ID No. 21 before inactivation, in particular consisted of the nucleotide sequence of SEQ ID No. 21 before inactivation.

Preferably, the at least one gene encoding erythritol isomerase 2 (Eyi2) of the genetically modified saprotroph comprised a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 21 before inactivation. In a preferred embodiment of the present invention, the at least one gene encoding erythritol isomerase 2 (Eyi2) of the genetically modified saprotroph consisted of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 21 before inactivation.

Preferably, the erythritol isomerase 2 (Eyi2) encoded by the nucleotide sequence of SEQ ID No. 21 before inactivation comprises the amino acid sequence of SEQ ID No. 22, in particular consists of the amino acid sequence of SEQ ID No. 22.

In a preferred embodiment, the erythritol isomerase 2 (Eyi2) encoded by the nucleotide sequence of SEQ ID No. 21 before inactivation comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 22. Particularly preferred, the erythritol isomerase 2 (Eyi2) encoded by the nucleotide sequence of SEQ ID No. 21 before inactivation consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 22.

Preferably, the erythritol isomerase 2 (Eyi2) encoded by the nucleotide sequence of SEQ ID No. 21 before inactivation comprises an amino acid sequence as defined in SEQ ID No. 22, in which one, preferably two, preferably three, preferably five, preferably six, preferably seven, preferably eight, preferably nine, preferably ten, amino acids are exchanged by other naturally occurring amino acids. In a preferred embodiment of the present invention, the erythritol isomerase 2 (Eyi2) encoded by the nucleotide sequence of SEQ ID No. 21 before inactivation comprises an amino acid sequence as defined in SEQ ID No. 22, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a further preferred embodiment of the present invention, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is deleted.

Preferably, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is non-functional.

In a particularly preferred embodiment, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is inactivated by gene knock-out, in particular gene replacement. Preferably, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is inactivated by gene replacement using a deletion cassette.

In a preferred embodiment of the present invention, the at least one further inactivated gene selected from the group consisting of a gene encoding phospho-2-dehydro-3-deoxyheptonate aldolase 1 (Dhaps-1), a gene encoding erythritol utilization factor (EUF), a gene encoding erythrulose kinase (EYK1), a gene encoding erythritol dehydrogenase (EYD1), a gene encoding erythritol isomerase (EYI1) and a gene encoding erythritol isomerase (EYI2) of the genetically modified saprotroph is inactivated, in particular made non-functional, by genome editing, in particular by meganucleases, zinc-finger nucleases (ZFNs), transcription activator-like effector nucleases (TALENs) or by the clustered regularly interspaced short palindromic repeats (CRISPR) system.

In a further preferred embodiment of the present invention, the genetically modified saprotroph further comprises at least one gene encoding at least one transketolase. Preferably, the at least one gene encoding at least one transketolase is tkl1, in particular is codon-optimized tkl1. Preferably, the genetically modified saprotroph further comprises at least one gene encoding tkl1 from T. reesei, in particular codon-optimized ikll from T. reesei. In a preferred embodiment, the at least one gene encoding at least one transketolase comprises the nucleotide sequence of SEQ ID No. 5, in particular consists of the nucleotide sequence of SEQ ID No. 5.

In a further preferred embodiment of the present invention, the at least one gene encoding at least one transketolase comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 5. Preferably, the at least one gene encoding at least one transketolase consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 5.

Preferably, the at least one transketolase comprises the amino acid sequence of SEQ ID No. 6, in particular consists of the amino acid sequence of SEQ ID No. 6.

In another preferred embodiment of the present invention, the at least one transketolase comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 6. Particularly preferred, the at least one transketolase consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 6.

In a further preferred embodiment of the present invention, the at least one transketolase comprises an amino acid sequence as defined in SEQ ID No. 6, in which one, preferably two, preferably three, preferably five, preferably six, preferably seven, preferably eight, preferably nine, preferably ten, amino acids are exchanged by other naturally occurring amino acids. In a particularly preferred embodiment, the at least one transketolase comprises an amino acid sequence as defined in SEQ ID No. 6, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the genetically modified saprotroph further comprises at least one gene encoding at least one transaldolase. Preferably, the at least one gene encoding at least one transaldolase is tall, in particular is codon-optimized tall. Preferably, the genetically modified saprotroph further comprises at least one gene encoding tall from T. reesei, in particular codon-optimized tall from T. reesei. In a preferred embodiment, the at least one gene encoding at least one transaldolase comprises the nucleotide sequence of SEQ ID No. 7, in particular consist of the nucleotide sequence of SEQ ID No. 7.

In another preferred embodiment, the at least one gene encoding at least one transaldolase comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 7. Preferably, the at least one gene encoding at least one transaldolase consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 7.

Preferably, the at least one transaldolase comprises the amino acid sequence of SEQ ID No. 8, in particular consists of the amino acid sequence of SEQ ID No. 8.

Particularly preferred, the at least one transaldolase comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 8. Preferably, the at least one transaldolase consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 8.

In another preferred embodiment of the present invention, the at least one transaldolase comprises an amino acid sequence as defined in SEQ ID No. 8, in which one, preferably two, preferably three, preferably five, preferably six, preferably seven, preferably eight, preferably nine, preferably ten, amino acids are exchanged by other naturally occurring amino acids. In a particularly preferred embodiment, the at least one transaldolase comprises an amino acid sequence as defined in SEQ ID No. 8, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a further preferred embodiment of the present invention, the genetically modified saprotroph further comprises at least one gene encoding at least one erythritol utilization factor (EUF). Preferably, the at least one gene encoding at least one erythritol utilization factor, is euf1, in particular is codon-optimized euf1. Preferably, the genetically modified saprotroph further comprises at least one gene encoding euf1 from T. reesei, in particular codon-optimized euf1 from T. reesei. In a preferred embodiment, the at least one gene encoding at least one erythritol utilization factor (EUF) comprises the nucleotide sequence of SEQ ID No. 13, in particular consist of the nucleotide sequence of SEQ ID No. 13.

In a preferred embodiment, the at least one gene encoding at least one erythritol utilization factor of the genetically modified saprotroph comprises a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 13. Preferably, the at least one gene encoding at least one erythritol utilization factor consists of a nucleotide sequence having at least 80%, preferably at least 85%, preferably at least 90%, preferably at least 95%, preferably at least 96%, preferably at least 97%, preferably at least 98%, preferably at least 99%, sequence identity to SEQ ID No. 13.

Preferably, the at least one erythritol utilization factor (EUF) comprises the amino acid sequence of SEQ ID No. 14, in particular consists of the amino acid sequence of SEQ ID No. 14.

In a further preferred embodiment, the at least one erythritol utilization factor comprises an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 14. Preferably, the at least one erythritol utilization factor consists of an amino acid sequence having at least 80%, preferably at least 85%, preferably at least 90%, preferably at least 95%, preferably at least 96%, preferably at least 97%, preferably at least 98%, preferably at least 99%, sequence identity to SEQ ID No. 14.

In a further preferred embodiment of the present invention, the at least one erythritol utilization factor comprises an amino acid sequence as defined in SEQ ID No. 14, in which one, preferably two, preferably three, preferably five, preferably six, preferably seven, preferably eight, preferably nine, preferably ten, amino acids are exchanged by other naturally occurring amino acids. In a particularly preferred embodiment, the at least one erythritol utilization factor comprises an amino acid sequence as defined in SEQ ID No. 14, in which at most ten, preferably at most nine, preferably at most eight, preferably at most seven, preferably at most six, preferably at most five, preferably at most four, preferably at most three, preferably at most two, preferably at most one, amino acids are exchanged by other naturally occurring amino acids.

In a preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) of the genetically modified saprotroph is an exogenous polynucleotide sequence.

Preferably, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) of the genetically modified saprotroph is an endogenous polynucleotide sequence.

In a further preferred embodiment, the genetically modified saprotroph comprises at least one exogenous gene encoding at least one membrane-bound alditol transporter. Preferably, genetically modified saprotroph comprises at least one endogenous gene encoding at least one membrane-bound alditol transporter.

Preferably, the genetically modified saprotroph comprises at least one exogenous gene encoding at least one erythrose reductase. In another preferred embodiment, the genetically modified saprotroph comprises at least one endogenous gene encoding at least one erythrose reductase.

Further preferred, the genetically modified saprotroph comprises at least one exogenous gene encoding at least one transketolase. In a preferred embodiment of the present invention, the genetically modified saprotroph comprises at least one endogenous gene encoding at least one transketolase.

Preferably, the genetically modified saprotroph comprises at least one exogenous gene encoding at least one transaldolase. Preferably, the genetically modified saprotroph comprises at least one endogenous gene encoding at least one transaldolase.

Preferably, the genetically modified saprotroph comprises at least one exogenous gene encoding at least one erythritol utilization factor (EUF). Preferably, the genetically modified saprotroph comprises at least one endogenous gene encoding at least one erythritol utilization factor (EUF).

In a preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) is stably or transiently introduced into the genome of the genetically modified saprotroph.

In a particularly preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF), is overexpressed.

Preferably, the at least one gene encoding at least one membrane-bound alditol transporter, in particular the at least one exogenous or endogenous gene encoding at least one membrane-bound alditol transporter, of the genetically modified saprotroph is overexpressed. In a further preferred embodiment, the at least one gene encoding at least one erythrose reductase, in particular the at least one exogenous or endogenous gene encoding at least one erythrose reductase, of the genetically modified saprotroph is overexpressed. Preferably, the at least one gene encoding at least one transketolase, in particular the at least one exogenous or endogenous gene encoding at least one transketolase, of the genetically modified saprotroph is overexpressed. Further preferred, the at least one gene encoding at least one transaldolase, in particular the at least one exogenous or endogenous gene encoding at least one transaldolase, of the genetically modified saprotroph is overexpressed. Preferably, the at least one gene encoding at least one erythritol utilization factor (EUF), in particular the at least one exogenous or endogenous gene encoding at least one erythritol utilization factor (EUF), of the genetically modified saprotroph is overexpressed.

In a further preferred embodiment of the present invention, the at least one gene encoding at least one membrane-bound alditol transporter, the at least one gene encoding at least one erythrose reductase, the at least one gene encoding at least one transketolase, the at least one gene encoding at least one transaldolase and/or the at least one gene encoding at least one erythritol utilization factor (EUF) of the genetically modified saprotroph is under the control of a constitutive or inducible promoter. In a preferred embodiment, the constitutive or inducible promoter is a naturally occurring promoter. In a further preferred embodiment, the constitutive or inducible promoter is a synthetic promoter. Preferably, the constitutive promoter is selected from pki, tef, gpd. The inducible promoter is preferably selected from bgal, bxl1, cbh1, cbh2, xyn1, xyn2. Preferably, the at least one gene of the genetically modified saprotroph encoding at least one membrane-bound alditol transporter is under the control of a promoter selected from pki, tef, gpd, preferably under control of a pki promoter. Preferably, the at least one gene of the genetically modified saprotroph encoding at least one erythrose reductase is under the control of a promoter selected from pki, tef, gpd, preferably under control of a tef promoter. Preferably, the at least one gene of the genetically modified saprotroph encoding at least one transketolase is under the control of a promoter selected from pki, tef, gpd, preferably under control of a tef promoter. Preferably, the at least one gene of the genetically modified saprotroph encoding at least one transaldolase is under the control of a promoter selected from pki, tef, gpd, preferably under control of a tef promoter. Preferably, the at least one gene of the genetically modified saprotroph encoding at least one erythritol utilization factor (EUF) is under the control of a promoter selected from pki, tef, gpd, preferably under control of a tef promoter.

In a further preferred embodiment of the present invention, the at least one gene of the genetically modified saprotroph encoding at least one membrane-bound alditol transporter, the at least one gene of the genetically modified saprotroph encoding at least one erythrose reductase, the at least one gene of the genetically modified saprotroph encoding at least one transketolase, the at least one gene of the genetically modified saprotroph encoding at least one transaldolase and/or the at least one gene of the genetically modified saprotroph encoding at least one erythritol utilization factor (EUF) is present on a plasmid. Preferably, each of the at least one gene of the genetically modified saprotroph encoding at least one membrane-bound alditol transporter, the at least one gene of the genetically modified saprotroph encoding at least one erythrose reductase, the at least one gene of the genetically modified saprotroph encoding at least one transketolase, the at least one gene of the genetically modified saprotroph encoding at least one transaldolase and/or the at least one gene of the genetically modified saprotroph encoding at least one erythritol utilization factor (EUF) is present on a separate plasmid.

In a particularly preferred embodiment of the present invention, the genetically modified saprotroph is the Trichoderma reesei strain deposited at the Westerdijk Fungal Biodiversity Institute under CBS number 146708.

According to a preferred embodiment of the present invention, the at least one saprotroph is cultivated in step a) in a volume of at least 5 L, preferably at least 10 L, preferably, at least 25 L, preferably at least 50 L, preferably at least 100 L, preferably at least 250 L, preferably at least 500 L, preferably at least 1.000 L, preferably at least 2.500 L, preferably at least 7.500 L, preferably at least 10.000 L, preferably at least 25.000 L, preferably at least 50.000 L, preferably at least 75.000 L, preferably at least 100.000 L.

In a further preferred embodiment of the present invention, the at least one saprotroph is cultivated in step a) in the culture medium until the culture medium contains erythritol in a concentration of at least 250 mg/L, preferably at least 300 mg/L, preferably at least 350 mg/L, preferably at least 400 mg/L, preferably at least 450 mg/L, preferably at least 500 mg/L, preferably at least 600 mg/L, preferably at least 700 mg/L, preferably at least 800 mg/L, preferably at least 900 mg/L, preferably at least 1 g/L, preferably at least 2 g/L, preferably at least 3 g/L, preferably at least 4 g/L, preferably at least 5 g/L, preferably at least 6 g/L, preferably at least 7 g/L, preferably at least 8 g/L, preferably at least 9 g/L, preferably at least 10 g/L, preferably at least 15 g/L, preferably at least 20 g/L, preferably at least 25 g/L, preferably at least 30 g/L, preferably at least 35 g/L, preferably at least 40 g/L.

Particularly preferred, the at least one saprotroph is cultivated in the culture medium until the culture medium contains erythritol in the concentration of 250 mg/L to 40 g/L, preferably 500 mg/L to 40 g/L, preferably 750 mg/L to 40 g/L, preferably 1 g/L to 40 g/L.

In a further preferred embodiment, the at least one saprotroph is cultivated in the culture medium until the culture medium contains erythritol in the concentration of 5 g/L to 40 g/L, preferably 10 g/L to 40 g/L, preferably 15 g/L to 40 g/L, preferably 20 g/L to 40 g/L, preferably 25 g/L to 40 g/L, preferably 30 g/L to 40 g//L.

According to a preferred embodiment of the present invention, the yield of erythritol is at least 3 mg/g, preferably at least 4 mg/g, preferably at least 5 mg/g, preferably at least 6 mg/g, preferably at least 7 mg/g, preferably at least 8 mg/g, preferably at least 9 mg/g, preferably at least 10 mg/g, preferably at least 11 mg/g, preferably at least 12 mg/g, preferably at least 13 mg/g, preferably at least 14 mg/g, preferably at least 15 mg/g, preferably at least 17.5 mg/g, preferably at least 20 mg/g, preferably at least 25 mg/g, preferably at least 30 mg/g, preferably at least 40 mg/g, preferably at least 50 mg/g, preferably at least 75 mg/g, preferably at least 100 mg/g (each based on mg erythritol/g carbon source consumed, in particular mg erythritol/g glucose consumed).

In a preferred embodiment, the yield of erythritol is 3 to 100 mg/g, preferably 4 to 100 mg/g, preferably 5 to 100 mg/g, preferably 6 to 100 mg/g, preferably 7 to 75 mg/g, preferably 8 to 75 mg/g, preferably 9 to 75 mg/g, preferably 10 to 75 mg/g, preferably 15 to 50 mg/g (each based on mg erythritol/g carbon source consumed, in particular mg erythritol/g glucose consumed).

In a particularly preferred embodiment of the present invention, the at least one saprotroph is cultivated in step a) in the culture medium at a pH in the range of 2 to 7, preferably 2.5 to 6.5, preferably 2.5 to 6, preferably 3 to 5.5. Particularly preferred the at least one saprotroph is cultivated in the culture medium at a pH of at most 7, preferably at most 6.5, preferably at most 6, preferably at most 5.5, preferably at most 5.

According to a preferred embodiment of the present invention, the at least one saprotroph is cultivated in step a) in the culture medium at a temperature of 25 to 35° C., preferably 26 to 34° C., preferably 27 to 33° C., preferably 28 to 32° C., preferably 29 to 31° C., preferably 30° C.

In a preferred embodiment of the present invention, the at least one saprotroph is cultivated in step a) in the culture medium at a temperature of at least 15° C., preferably at least 20° C., preferably at least 25° C., preferably at least 26° C., preferably at least 27° C., preferably at least 28° C.

In a further preferred embodiment, the at least one saprotroph is cultivated in step a) in the culture medium at a temperature of at most 35° C., preferably at most 34° C., preferably at most 33° C., preferably at most 32° C., preferably at most 31° C.

Preferably, the at least one saprotroph is cultivated in the culture medium in step a) for at least 24 hours, preferably at least 36 hours, preferably at least 48 hours, preferably at least 60 hours, preferably at least 72 hours, preferably at least 84 hours, preferably at least 96 hours, preferably at least 108 hours, preferably at least 120 hours, preferably at least 132 hours, preferably at least 144 hours, preferably at least 156 hours, preferably at least 168 hours.

In a further preferred embodiment of the present invention, the at least one saprotroph is cultivated in the culture medium in step a) for at most 240 hours, preferably at most 216 hours, preferably at most 192 hours, preferably at most 180 hours, preferably at most 168 hours, preferably at most 156 hours, preferably at most 144 hours, preferably at most 132 hours, preferably at most 120 hours, preferably at most 108 hours, preferably at most 96 hours, preferably at most 84 hours, preferably at most 72 hours.

According to a preferred embodiment of the present invention, the cultivation of the at least one saprotroph in step a) is conducted without the addition of osmotically effective agents during cultivation, in particular without the addition of at least one salt during cultivation. Particularly preferred, the cultivation of the at least one saprotroph in step a) is conducted without the addition of sodium chloride (NaCl) during cultivation.

In a preferred embodiment of the present invention, the cultivation of the at least one saprotroph in step a) is conducted in batch-mode. Preferably, the cultivation of the at least one saprotroph in step a) is conducted in fed-batch mode. In another preferred embodiment, cultivation of the at least one saprotroph in step a) is conducted in continuous mode.

In a further preferred embodiment of the present invention the erythritol is recovered in step c) by crystallisation.

In a particularly preferred embodiment of the present invention, the recovery of erythritol in step b) comprises the steps:
i) removal of biomass, preferably removal of biomass by centrifugation and membrane filtration,
ii) decolorisation of the culture medium, preferably decolorisation of the culture medium with active carbon,
iii) desalting the culture medium, preferably desalting and decolorizing the culture medium by electrodialysis,
iv) preparative chromatography, preferably reverse phase chromatography using ion exclusion and size exclusion mechanisms, and
v) concentrating and crystalizing the erythritol.

According to a preferred embodiment of the present invention, a heat denaturing step is conducted prior to, during or after step i). Preferably, a heat denaturing step is conducted prior to step i). In a further preferred embodiment, a heat denaturing step is conducted during step i). Preferably, a heat denaturing step is conducted after step i).

Particularly preferred, the heat denaturing step includes heating the culture medium to a temperature of at least 45° C., preferably at least 50° C., preferably at least 55° C., preferably at least 60° C., preferably at least 65° C., preferably at least 70° C., preferably 75° C., preferably at least 80° C., preferably at least 85° C., preferably at least 90° C., preferably at least 95° C.

In a preferred embodiment of the present invention, the culture medium is subjected to heat denaturation for at least 10 seconds, preferably at least 20 seconds, preferably at least 30 seconds, preferably at least 40 seconds, preferably at least 50 seconds, preferably at least 1 min, preferably at least 2 min, preferably at least 3 min, preferably at least 4 min, preferably at least 5 min, preferably at least 6 min, preferably at least 7 min, preferably at least 8 min, preferably at least 9 min, preferably at least 10 min.

The heat denaturing step primarily serves to heat-inactivate proteins and enzymes in the culture medium before conducting further recovery steps.

In a particularly preferred embodiment of the present invention, the erythritol recovered in step b), in particular the erythritol obtained in step v), has a purity of at least 90%, preferably at least 92%, preferably at least 94%, preferably at least 96%, preferably at least 97%, preferably at least 97.5%, preferably at least 98%, preferably at least 98.5%, preferably at least 99%, preferably at least 99.5%.

The term “genetically modified” and its grammatical equivalents as used herein refer to one or more alterations of a nucleic acid, e.g. the nucleic acid within the genome of an organism, in particular within the genome of a saprotroph. A “genetically modified” saprotroph can refer to a saprotroph with an added, deleted and/or altered, in particular inactivated, gene.

In the context of the present invention, the term “erythrose reductase” pertains to any enzyme that catalyses reversibly the reduction of an aldose to an alditol, wherein the enzyme has predominant specific activity for the reduction of erythrose to erythritol. Thus, an “erythrose reductase” according to the present invention exhibits a high specificity and affinity for the substrate erythrose. Particularly, the “erythrose reductase” has a higher specificity for erythrose than for any other substrate, such as glycerol.

The term “membrane-bound alditol transporter” as used herein designates a membrane protein suitable to reversibly transport alditols across the outer membrane, in particular to transport intracellularly produced alditols to the culture medium. According to the present invention, a “membrane-bound alditol transporter” is suitable to transport intracellularly produced erythritol across the membrane to the culture medium.

In the context of the present invention, the term “transketolase” refers to an enzyme of the pentose phosphate pathway catalysing the thiamine-dependent reversible transfer of a C-2 unit from a ketose donor to an acceptor aldopentose. In particular, the “transketolase” catalyses the transfer of a C-2 unit from xylulose-5-phosphate to ribose-5-phosphate forming seduheptulose-7-phosphate and glyceraldehyde-3-phosphate.

The term “transaldolase” designates an enzyme of the non-oxidative branch of the pentose phosphate pathway catalysing the reversible transfer of a C-3 unit from a ketose donor to an acceptor aldose. In particular, the “transketolase” catalyses the transfer of a C-3 unit from sedoheptulose-7-phosphate to glyceraldehyde-3-phosphate forming erythrose-4-phosphate and fructose-6-phosphate.

In the context of the present invention, an “inactivated” gene is a gene which can temporarily or permanently not be transcribed or can be transcribed but the transcript is not accessible for gene translation. According to the present invention, the inactivation of a gene includes but is not limited to inactivation by partial or complete gene deletion, partial or complete gene replacement, gene repression, gene insertion, and gene mutation. “Inactivation” in the context of the present invention further includes any post-transcriptional gene regulation, in particular by RNAi or siRNA, resulting in inhibition of translation of the transcript into the gene product.

In the context of the present invention, a “non-functional” gene is a gene that is present in the genome of an organism or a plasmid in an organism which gene is either not transcribed or is transcribed but the transcript is not translated to the gene product.

In the context of the present invention, a “deleted” gene is a gene that has previously been present in the genome of an organism or a plasmid in an organism but has been completely removed from the genome of the organism or the plasmid in the organism.

In the context of the present invention, the verb “to overexpress” refers to the artificial expression of a gene in increased quantity. This includes the inducible overexpression of a gene which can be regulated using an inducible promoter and the constant overexpression of a gene using a constitutive promoter.

The term “oligomeric sugars” or “oligomeric C-5 and C-6 sugars” is used herein for di-, oligo-, and polysaccharides which are composed of at least two monomers, preferably composed of at least two monomers selected from C-5 and C-6 sugars.

In the context of the present invention, the term “a” is meant to include the meaning of “one” or “one or more”.

In the context of the present invention, the term “comprising” preferably has the meaning of “containing” or “including”, meaning including the specifically identified elements without excluding the presence of further elements. However, in a preferred embodiment, the term “comprising” is also understood to have the meaning of “consisting essentially of” and in a further preferred embodiment the meaning of “consisting of”.

The terms “and/or” is used herein to express the disclosure of any combination of individual elements connected within a listing. Solely for illustrative purposes, the expression “A, B, and/or C” can mean A individually, B individually, C individually, (A and B), (B and C), (A and C), and (A, B and C).

Further preferred embodiments of the invention are subject of the subclaims.

The invention is further described by way of the following example and accompanying figures.

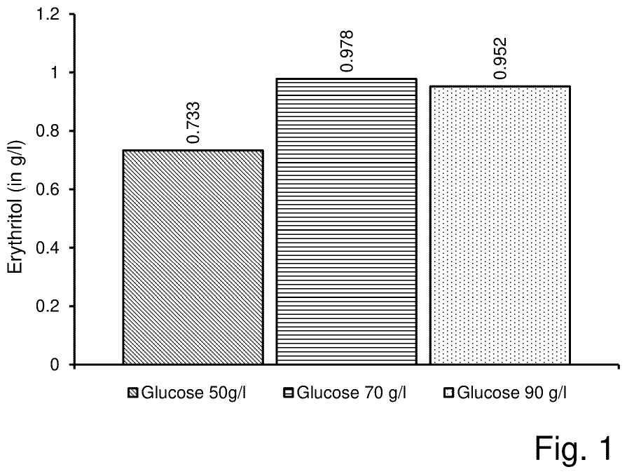

FIG. 1 shows the influence of the carbon source concentration in the culture medium on erythritol production by Trichoderma reesei after 168 hours incubation in a culture medium comprising either 50 g/L, 70 g/L or 90 g/L glucose as carbon source and 80 mM urea as nitrogen source.

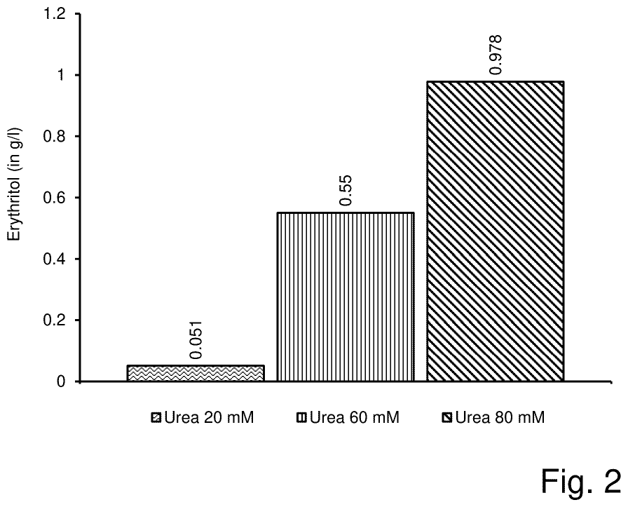

FIG. 2 shows the influence of the nitrogen source concentration in the culture medium on erythritol production by Trichoderma reesei after 168 hours incubation in a culture medium comprising 70 g/L glucose as carbon source and either 20 mM, 60 mM or 80 mM urea as nitrogen source.

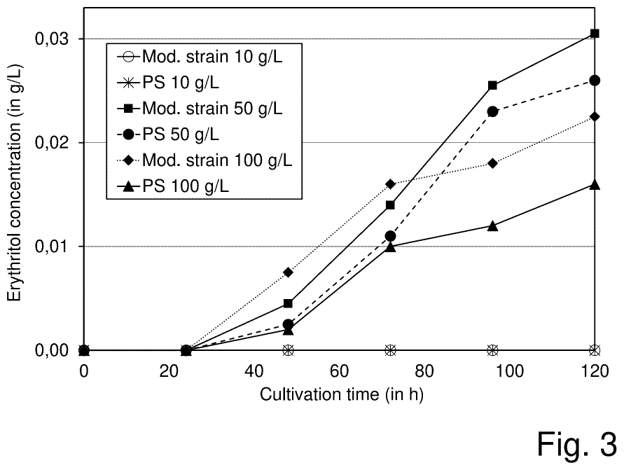

FIG. 3 illustrates the influence of the initial carbon source concentration on erythritol production by the genetically modified strain QM6aΔtmus53 fps1 (Reasl1) err1 (Repyr4) Ampdh (“Mod. strain”) and by the corresponding parental strain QM6aΔtmus53 (“PS”) after 24, 48, 72, 96 and 120 hours of incubation in a culture medium comprising either 10 g/L, 50 g/L or 100 g/L glucose as carbon source and 20 mM ammonium sulfate as nitrogen source.

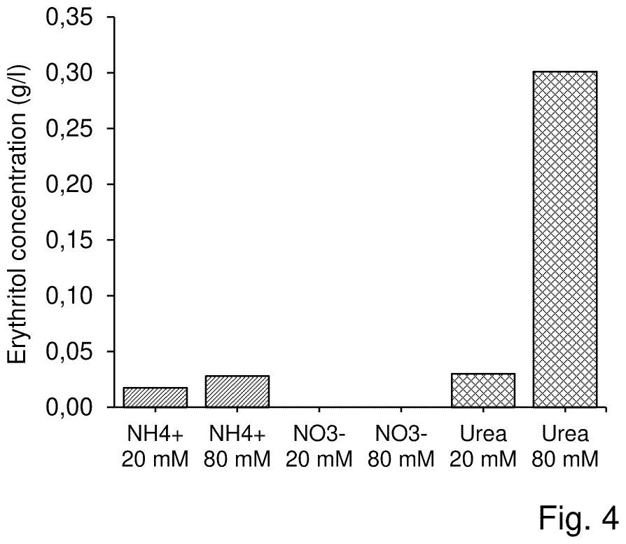

FIG. 4 shows the effect of different nitrogen sources in a concentration of 20 mM or 80 mM on the erythritol production of Trichoderma reesei (QM6aΔtmus53) after 96 hours of cultivation in a culture medium comprising 50 g/L glucose as carbon source.

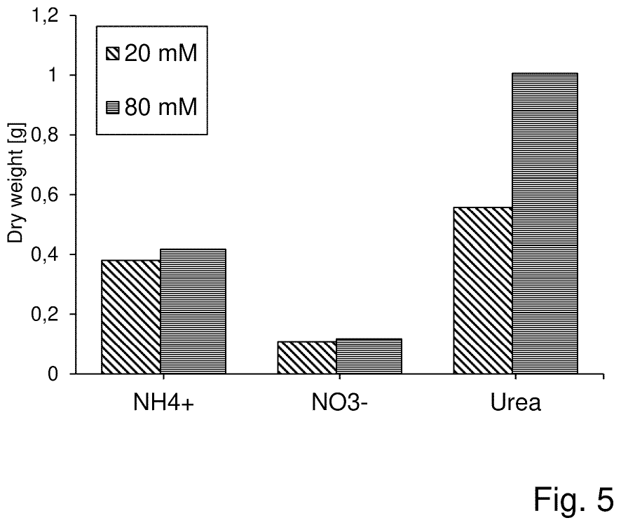

FIG. 5 shows the influence of the nitrogen source on biomass generation (dry weight) of Trichoderma reesei (QM6aΔtmus53) after 96 hours of cultivation in a culture medium comprising 50 g/L glucose as carbon source and 20 mM or 80 mM of the respective nitrogen source.

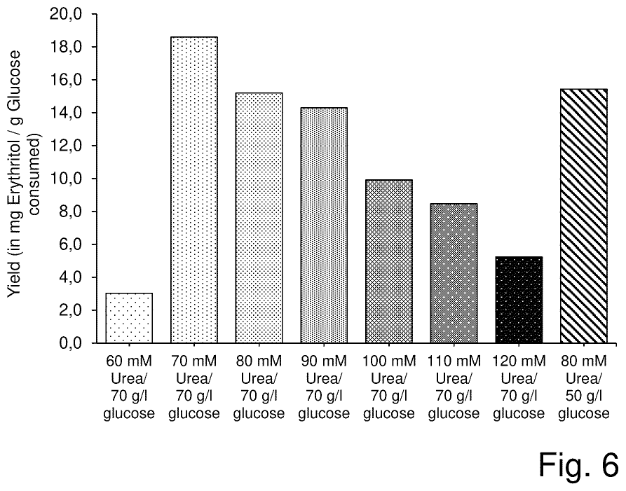

FIG. 6 illustrates the influence of the urea concentration on the erythritol production of Trichoderma reesei (QM6aΔtmus53) after 168 hours cultivation in a culture medium comprising 70 g/L glucose and 60 mM, 70 mM, 80 mM, 90 mM, 100 mM, 110 mM or 120 mM urea or in a culture medium comprising 50 g/L glucose and 80 mM urea (right bar).

### EXAMPLE

### 1. Biotechnological Production of Erythritol

### 1.1 Influence of the Carbon Source Concentration on Erythritol Production

Trichoderma reesei was cultivated in 250 mL flasks each comprising 100 mL culture medium containing either 50 g/L, 70 g/L or 90 g/L glucose as carbon source and 80 mM urea as nitrogen source.

After 168 hours of cultivation, the erythritol concentration was measured in the culture medium of the samples. The average concentration of erythritol in the samples was 0.733 g/L (50 g/L glucose), 0.978 g/L (70 g/L glucose), and 0.952 g/L (90 g/L glucose) (see FIG. 1).

### 1.2 Influence of the Nitrogen Source Concentration on Erythritol Production

In a similar experiment, Trichoderma reesei was cultivated in 250 mL flasks each comprising 100 mL culture medium containing 70 g/L glucose as carbon source and either 20 mM, 60 mM or 80 mM urea as nitrogen source.

After 168 hours of cultivation, the erythritol concentration in the culture medium of the samples was measured. The average concentration of erythritol in the samples was 0.051 g/L (20 mM urea), 0.55 g/L (60 mM urea), and 0.978 g/L (80 mM urea) (see FIG. 2).

### 1.3 Influence of the Initial Glucose Concentration on Erythritol Production by a Genetically Modified Strain and the Corresponding Parental Strain

Trichoderma reesei strains QM6aΔtmus53 fps1 (Reasl1) err1 (Repyr4) Ampdh (“Mod. strain”) and the corresponding parental strain QM6aΔtmus53 (“PS”) were cultivated in 250 mL flasks each comprising 100 mL culture medium containing either 10 g/L, 50 g/L or 100 g/L glucose as carbon source and 20 mM ammonium sulfate as nitrogen source.

After 24, 48, 72, 96, and 120 hours of cultivation, the erythritol concentration was measured in the culture medium of the samples (see table 1 and FIG. 3). No erythritol could be detected in culture medium containing only 10 g/L glucose.

TABLE 1
Erythritol concentration

| | | Erythritol concentration [g/L] | | | | | |
|---|---|---|---|---|---|---|---|
| | | | Parental | | Parental | | Parental |
| | | Mod. | strain | Mod. | strain | Mod. | strain |
| Incubation | | strain | (PS) | strain | (PS) | strain | (PS) |
| time | | 10 g/L | 10 g/L | 50 g/L | 50 g/L | 100 g/L | 100 g/L |
| 24 | h | 0 | 0 | 0 | 0 | 0 | 0 |
| 48 | h | 0 | 0 | 0.0045 | 0.0025 | 0.0075 | 0.002 |
| 72 | h | 0 | 0 | 0.014 | 0.011 | 0.016 | 0.01 |
| 96 | h | 0 | 0 | 0.0255 | 0.0.023 | 0.018 | 0.012 |
| 120 | h | 0 | 0 | 0.0305 | 0.026 | 0.0225 | 0.016 |

1.4 Influence of Different Nitrogen Sources on the Erythritol Production by Trichoderma reesei

To investigate the effect of different nitrogen sources on the erythritol production of Trichoderma reesei, the strain QM6aΔtmus53 was cultivated in 250 mL flasks comprising 100 mL of a culture medium with 50 g/L glucose as carbon source and 20 mM or 80 mM of either ammonium sulfate, sodium nitrate, or urea as nitrogen source.

After 96 hours of cultivation, the erythritol concentration was measured in the culture medium of the samples. The average concentration of erythritol in the samples was 0.018 g/L (50 g/L glucose, 20 mM NH₄⁺), 0.028 g/L (50 g/L glucose, 80 mM NH₄⁺), 0.03 g/L (50 g/L glucose, 20 mM urea), 0.301 g/L (50 g/L glucose, 80 mM urea). No erythritol could be detected in the culture medium after 96 hours in case 20 or 80 mM NO₃⁻ was used as nitrogen source (see FIG. 4).

1.5 Influence of Different Nitrogen Sources on the Growth of Trichoderma reesei

Trichoderma reesei (QM6aΔtmus53) was cultivated in 250 mL flasks each comprising 100 mL culture medium containing 50 g/L glucose as carbon source and 20 mM or 80 mM of either ammonium sulfate, sodium nitrate, or urea as nitrogen source.

After 96 hours of cultivation, the dry weight of Trichoderma reesei was measured in the different samples. The average weight was 0.384 g (50 g/L glucose, 20 mM NH₄⁺), 0.417 g/L (50 g/L glucose, 80 mM NH₄⁺), 0.107 g (50 g/L glucose, 20 mM NO₃⁻), 0.117 g (50 g/L glucose, 80 mM NO₃⁻), 0.557 g (50 g/L glucose, 20 mM urea), and 1.007 g (50 g/L glucose, 80 mM urea) (see FIG. 5).

### 1.6 Effect of the Urea Concentration in the Culture Medium on the Yield of Erythritol

The influence of the urea concentration in the culture medium on the yield of erythritol in reference to the amount of glucose consumed by Trichoderma reesei (QM6aΔtmus53) during cultivation was investigated. For this purpose, Trichoderma reesei was cultivated in 250 mL flasks comprising 100 mL of a culture medium with 70 g/L glucose as carbon source and either 60 mM, 70 mM, 80 mM, 90 mM, 100 mM, 110 mM or 120 mM urea as nitrogen source. The yield of erythritol was also measured for the cultivation of Trichoderma reesei in the presence of 100 mL culture medium comprising 50 g/L glucose as carbon source and 80 mM urea as nitrogen source.

After 168 hours of cultivation, the erythritol concentration in the culture medium and the decrease in glucose concentration were measured in the different samples. The average yield of erythritol (in mg erythritol/g glucose consumed) was 3.03 mg/g (70 g/L glucose, 60 mM urea), 18.59 mg/g (70 g/L glucose, 70 mM urea), 15.2 mg/g (70 g/L glucose, 80 mM urea), 14.3 mg/g (70 g/L glucose, 90 mM urea), 9.92 mg/g (70 g/L glucose, 100 mM urea), 8.47 mg/g (50 g/L glucose, 110 mM urea), 5.23 mg/g (70 g/L glucose, 120 mM urea) and 15.43 mg/g (50 g/L glucose, 80 mM urea) (see FIG. 6).

1. A method for the production of erythritol, comprising the steps:
a) cultivating at least one saprotroph in a culture medium comprising a carbon source in a concentration of at least 40 g/L and a nitrogen source, so as to obtain erythritol in the culture medium,
b) recovering erythritol from the culture medium.

2. The method according to claim 1, wherein the culture medium comprises the carbon source in a concentration of 40 to 200 g/L, preferably 70 to 90 g/L.

3. The method according to claim 1 or 2, wherein the culture medium comprises the nitrogen source in a concentration of at least 50 mM, preferably at least 60 mM, preferably at least 70 mM.

4. The method according to any one of the preceding claims, wherein the culture medium comprises a lignocellulose comprising hydrolysate.

5. The method according to any one of the preceding claims, wherein the carbon source is glucose or xylose.

6. The method according to any one of claims 1 to 4, wherein the carbon source comprises a hydrolysate obtained by hydrothermal treatment of a cellulose-, hemi-cellulose- and/or starch-comprising raw material.

7. The method according to any one of the preceding claims, wherein the nitrogen source is urea.

8. The method according to any one of the preceding claims, wherein the at least one saprotroph is a filamentous fungus from the genus Hypocrea, Trichoderma, Gibberella, Fusarium, Aspergillus or Penicillium.

9. The method according to any one of the preceding claim 1, wherein the saprotroph is selected from the group consisting of Hypocrea jecorina (Trichoderma reesei), Gibberella zeae, Fusarium graminearum, Aspergillus niger, Aspergillus nidulans, Aspergillus oryzae and Penicillium chrysogenum.

10. The method according to any one of the preceding claims, wherein the saprotroph is Hypocrea jecorina (Trichoderma reesei)

11. The method according to any one of the preceding claims, wherein in step a) the at least one saprotroph is cultivated in the culture medium until the culture medium contains erythritol in a concentration of at least 250 mg/L, preferably at least 500 mg/L, preferably at least 1 g/L.

12. The method according to any one of the preceding claims, wherein in step a) the at least one saprotroph is cultivated in the culture medium at a pH in the range of 2 to 7, preferably 3 to 5.5.

13. The method according to any one of the preceding claims, wherein the erythritol is recovered in step b) by crystallisation.

14. The method according to any one of the preceding claims, wherein the recovery of erythritol in step b) comprises the steps:
i) removal of biomass, preferably removal of biomass by centrifugation and membrane filtration,
ii) decolorisation of the culture medium, preferably decolorisation of the culture medium with active carbon,
iii) desalting the culture medium, preferably desalting and decolorizing the culture medium by electrodialysis,
iv) preparative chromatography, preferably reverse phase chromatography using ion exclusion and size exclusion mechanisms, and
v) concentrating and crystallising the erythritol.

15. The method according to claim 14, wherein a heat denaturing step is conducted prior to, during or after step i).
